SEQUENCE LISTING (40 sequences)

SEQ ID NO: 1 - RNA, 19 nt, Artificial Sequence
  HPV type 18 siRNA 426 Forward
caaccgagcacgacaggaa

SEQ ID NO: 2 - RNA, 19 nt, Artificial Sequence
  HPV type 18 siRNA 426 Reverse
uuccugucgugcucgguug

SEQ ID NO: 3 - RNA, 19 nt, Artificial Sequence
  HPV type 18 siRNA 450 Forward
ccaacgacgcagagaaaca

SEQ ID NO: 4 - RNA, 19 nt, Artificial Sequence
  HPV type 18 siRNA 450 Reverse
uguuucucugcgucguugg

SEQ ID NO: 5 - RNA, 19 nt, Artificial Sequence
  HPV type 16 siRNA 366 Forward
gcaaagacaucuggacaaa

SEQ ID NO: 6 - RNA, 19 nt, Artificial Sequence
  HPV type 16 siRNA 366 Reverse
uuuguccagaugucuuugc

SEQ ID NO: 7 - RNA, 19 nt, Artificial Sequence
  HPV type 16 siRNA 488 Forward
ucaagaacacguagagaaa

SEQ ID NO: 8 - RNA, 19 nt, Artificial Sequence
  HPV type 16 siRNA 488 Reverse
uuucucuacguguucuuga

SEQ ID NO: 9 - RNA, 21 nt, Artificial Sequence
  HPV type 16 siRNA 497 Forward
gaccggucgauguaugucuug

SEQ ID NO: 10 - RNA, 21 nt, Artificial Sequence
  HPV type 16 siRNA 497 Reverse
agacauacaucgaccggucca

SEQ ID NO: 11 - DNA, 21 bp, Artificial Sequence
  CDKN1A forward
cgaagtcagttccttgtggag

SEQ ID NO: 12 - DNA, 19 bp, Artificial Sequence
  CDKN1A reverse
catgggttctgacggacat

SEQ ID NO: 13 - DNA, 8 bp, Artificial Sequence
  TaqMan probe
cagaggag

SEQ ID NO: 14 - DNA, 23 bp, Artificial Sequence
  APAF1 forward
cctgttgtctcttcttccagtgt

SEQ ID NO: 15 - DNA, 20 bp, Artificial Sequence
  APAF1 reverse
aaaacaactggcctctgtgg

SEQ ID NO: 16 - DNA, 8 bp, Artificial Sequence
  TaqMan probe
aggtggag

SEQ ID NO: 17 - DNA, 18 bp, Artificial Sequence
  BAX forward
gaaccatcatgggctgga

SEQ ID NO: 18 - DNA, 20 bp, Artificial Sequence
  BAX reverse
cgtcccaaagtaggagagga

SEQ ID NO: 19 - DNA, 8 bp, Artificial Sequence
  TaqMan probe
cttcctcc

SEQ ID NO: 20 - DNA, 19 bp, Artificial Sequence
  PML forward
gagccccgtcataggaagt

SEQ ID NO: 21 - DNA, 18 bp, Artificial Sequence
  PML reverse
cacaacgcgttcctctcc

SEQ ID NO: 22 - DNA, 8 bp, Artificial Sequence
  TaqMan probe
gcaggaag

SEQ ID NO: 23 - DNA, 20 bp, Artificial Sequence
  YPEL3 forward
aaccacgacgacctcatctc

SEQ ID NO: 24 - DNA, 18 bp, Artificial Sequence
  YPEL3 reverse
agcccacgttcaccactg

SEQ ID NO: 25 - DNA, 8 bp, Artificial Sequence
  TaqMan probe
ccagggca

SEQ ID NO: 26 - DNA, 18 bp, Artificial Sequence
  GADD45A forward
ccccgataacgtggtgtt

SEQ ID NO: 27 - DNA, 19 bp, Artificial Sequence
  GADD45A reverse
gccacatctctgtcgtcgt

SEQ ID NO: 28 - DNA, 8 bp, Artificial Sequence
  TaqMan probe
gcctgctg

SEQ ID NO: 29 - DNA, 21 bp, Artificial Sequence
  XPC forward
agaccataccagagcccattt

SEQ ID NO: 30 - DNA, 20 bp, Artificial Sequence
  XPC reverse
aggctggtccatgtgttttg

SEQ ID NO: 31 - DNA, 8 bp, Artificial Sequence
  TaqMan probe
gggagaag

SEQ ID NO: 32 - DNA, 21 bp, Artificial Sequence
  PPM1D (Wip1) forward
cccatgttctacaccaccagt

SEQ ID NO: 33 - DNA, 22 bp, Artificial Sequence
  PPM1D (Wip1) reverse
tggtccttagaattcacccttg

SEQ ID NO: 34 - DNA, 8 bp, Artificial Sequence
  TaqMan probe
tggaggag

SEQ ID NO: 35 - DNA, 26 bp, Artificial Sequence
  MDM2 forward
ccatgatctacaggaacttggtagta

SEQ ID NO: 36 - DNA, 24 bp, Artificial Sequence
  MDM2 reverse
tcactcacagatgtacctgagtcc

SEQ ID NO: 37 - DNA, 8 bp, Artificial Sequence
  TaqMan probe
tcctgctg

SEQ ID NO: 38 - DNA, 24 bp, Artificial Sequence
  HPRT1 forward
tgaccttgatttattttgcatacc

SEQ ID NO: 39 - DNA, 20 bp, Artificial Sequence
  HPRT1 reverse
cgagcaagacgttcagtcct

SEQ ID NO: 40 - DNA, 8 bp, Artificial Sequence
  TaqMan probe
gctgagga

(19) United States
(12) Patent Application Publication
(10) Pub. No.: US 2018/0153932 A1
(43) Pub. Date: Jun. 7, 2018

(54) METHOD FOR MAINTAINING INCREASED INTRACELLULAR p53 LEVEL, INDUCED BY PLATINUM-BASED ANTICANCER DRUG, AND APPLICATION THEREOF

(71) Applicants: ABION INC., Seoul (KR); Seoul National University R&DB Foundation, Seoul (KR)

(72) Inventors: Young Kee SHIN, Seoul (KR); Young Deug KIM, Incheon (KR); Hun Soon JUNG, Seoul (KR); Deuk Ae KIM, Seoul (KR); Nirmal Rajasekaran, Seoul (KR)

(21) Appl. No.: 15/785,273

(22) Filed: Oct. 16, 2017

Related U.S. Application Data
(63) Continuation-in-part of application No. PCT/KR2016/004023, filed on Apr. 18, 2016.
(60) Provisional application No. 62/148,403, filed on Apr. 16, 2015.

(51) Int. Cl. A61K 33/24; C12N 15/113; A61K 31/7088; A61P 35/00; C12Q 1/6886

(52) U.S. Cl. CPC A61K 33/24; C12N 15/1137; A61K 31/7088; C12Q 2600/136; C12Q 1/6886; C12N 2310/14; C12N 2320/31; A61P 35/00

(57) ABSTRACT

The present invention relates to a method for maintaining the increased intracellular p53 level, induced by a platinum-based anticancer drug, and an application thereof and, more specifically, to a method for maintaining the increased intracellular p53 level in cells by administering a platinum-based anticancer drug and siRNA to ubiquitin ligase for p53 to a subject in need thereof in combination and sequentially, and a composition for promoting cancer cell apoptosis using the same.

According to the method of the present invention, the increased intracellular p53 expression level can be maintained for a long period of time in spite of the treatment with a low-concentration platinum-based anticancer drug, thereby effectively inducing the apoptosis of cancer cells and minimizing the drug side effect caused by the administration of the platinum-based anticancer drug, and thus the present invention can be favorably used in the prevention of cancer or the development of cancer medicines.

This application claims priority from U.S. Patent Application No. 62/148,403, filed Apr. 16, 2015, which is incorporated herein by reference in its entirety.

### TECHNICAL FIELD

The present invention relates to a method for maintaining an increased intracellular p53 level, induced by platinum-based anticancer drug, and application thereof, and more particularly, to a method for maintaining an increased level of p53 in cells by administering a platinum-based anticancer drug and an siRNA against ubiquitin ligase to p53 to a subject in need thereof, in combination or sequentially, and a composition for promoting the death of cancer cells using the same.

### BACKGROUND OF THE INVENTION

The tumor suppressor protein p53 plays a pivotal role in maintaining genome integrity in cells by regulating the expression of various arrays of genes responsible for DNA repair, cell cycle and growth arrest, and apoptosis [references [May et al., Oncogene 18 (53) (1999) p. 7621-7636]; [Oren, Cell Death Differ. 10 (4) (2003) p. 431-442]; [Hall and Peters, Adv. Cancer Res., 68: (1996) p. 67-108]; [Hainaut et al., Nucleic Acid Res., 25: (1997) p. 151-157]; [Sherr, Cancer Res, 60: (2000) p. 3689-95]]. In response to oncogenic stress signals, the cell activates the p53 transcription factor to activate genes involved in cell cycle regulation, thereby initiating apoptosis or cell cycle arrest. Apoptosis facilitates the removal of damaged cells from organisms, while cell cycle arrest allows damaged cells to repair genetic damage [references [Ko et al, Genes Dev. 10: (1996) p. 1054-1072]; [Levine, Cell 88: (1997) p. 323-331]]. The loss of the safeguard function of p53 makes it easy for damaged cells to progress to the cancerous state. Inactivation of p53 in mice produces an uncommonly high proportion of tumors consistently [references [Donehower et al, Nature, 356: (1992) p. 215-221]].

The p53 transcription factor facilitates the expression of a number of cell cycle control genes, including a gene encoding the human double minute 2 (HDM2) protein, a unique negative regulator [references [Chene, Nature Reviews Cancer 3: (2003) p. 102-109]; [Momand, Gene 242 (1-2): (2000) p. 15-29]; [Zhele va et al. Mini. Rev. Med. Chem. 3 (3): (2003) p. 257-270]]. The HDM2 protein acts to downregulate p53 activity in an autoregulated manner [references [Wu et al, Genes Dev, 7: (1993) p. 1126-1132]; [Bairak et al, EMBO J, 12: (1993) p. 461-468]]. Under the absence of tumorigenic stress signal, that is, under normal cell conditions, the HDM2 protein helps to keep p53 activity at a low level [references [Wu et al, Genes Dev, 7: (1993) p. 1126-1132]; [Bairak et al, EMBO J, 12: (1993) p. 461-468]]. However, in response to cellular DNA damage or under cell stress, p53 activity is increased to help prevent the proliferation of permanently damaged cell clones by induction of cell cycle and growth arrest or apoptosis.

The regulation of p53 function depends on a proper balance between the two components of the p53-HDM2 auto-regulating system. In fact, this balance appears to be essential for cell survival. There are at least three ways in which HDM2 acts to down-regulate p53 activity. First, HDM2 can bind to the N-terminal transcriptional activation domain of p53 and block the expression of the p53-reactive gene [references [Kussie et al, Science, 274: (1996) p. 948-953]; [Oliner et al, Nature, 362: (1993) p. 857-860]; [Momand et al, Cell, 69: (1992) p. 1237-1245]]. Second, HDM2 transports p53 back and forth from the nucleus to the cytoplasm to facilitate proteolytic hydrolysis of p53 [references [Roth et al, EMBO J, 17: (1998) p. 554-564]; [Freedman et al, Mol Cell Biol, 18: (1998) p. 7288-7293]; [Tao and Levine, Proc. Natl. Acad. Sci. 96: (1999) p. 3077-3080]]. Finally, HDM2 retains its intrinsic E3 ligase activity by conjugating ubiquitin to p53 for degradation in the ubiquitin-dependent 26S proteasome pathway [references [Honda et al, FEBS Lett, 420: (1997) p. 25-27]; [Yasuda, Oncogene 19: (2000) p. 1473-1476]]. Thus, HDM2 interferes with the capability of the p53 transcription factor, which is able to bind to p53 in the nucleus and thereby promote expression of the target gene. Weakening the p53-HDM2 auto-regulatory system can have a significant impact on cell homeostasis. Consistently, a correlation between overexpression of HDM2 and tumor formation has been reported [references [Chene, Nature 3: (2003) p. 102-109]. Functional inactivation of wild-type p53 is found in many types of human tumors. Restoring p53 function in tumor cells by anti-HDM2 therapy will result in slowing tumor growth and instead stimulate apoptosis. Naturally, there is now a substantial effort to identify novel anticancer agents that subsequently interfere with the ability of HDM2 to interact with p53 [references [Chene, Nature 3: (2003) p. 102-109]. It has been shown that antibodies, peptides, and antisense oligonucleotides disrupt the p53-HDM2 interaction, and this leads to the activation of the p53 pathway, which allows p53 to be released from the negative control of HDM2, allowing growth arrest and/or the normal signal of apoptosis to function, and this provides a potential therapeutic approach for the treatment of cancer, and other diseases characterized by abnormal cell proliferation [references [Blaydes et al, Oncogene 14: (1997) p. 1859-1868]; [Bottger et al, Oncogene 13 (10): (1996) p. 2141-2147]].

On the other hand, cisplatin among platinum-based anticancer drugs is known to be very effective in treating various cancers such as head and neck cancer, lung cancer, breast cancer, bladder cancer, stomach cancer, cervical cancer and myeloma. Cisplatin is a heavy metal compound containing platinum and has two chlorine atoms and two ammonia molecules in a cis-form centered on a platinum atom, and the cisplatin binds to two adjacent guanines on the DNA strand to forms an interstrand crosslink to inhibit DNA synthesis. That is, it is known that the anticancer effect is attained by attaching to the DNA double helix structure existing in the nucleus of cancer cells and inhibiting DNA replication, resulting in inhibiting the growth and proliferation of cancer cells and removing cancer cells. It is known that the expression of p53 is very important for such cisplatin to exert cytotoxicity against cancer cells (Apoptosis. 2007 September; 12(9):1733-42.), and there is a limitation in that the anticancer effect of cisplatin does not show a consistent pattern because the expression dynamics of p53 against cisplatin treatment are different depending on the kind of cell.

Apoptosis induced by increased expression of p53 is one of the most important therapeutic strategies in anticancer therapy. Therefore, if the increase of intracellular p53 expression by platinum-based anticancer drug can be sustained for a long time, it will be possible to treat cancer more effectively.

### DETAILED DESCRIPTION OF THE INVENTION

### [Technical Assignment]

Accordingly, the inventors of the present invention have made a careful effort to develop a therapeutic strategy in which the expression of p53 should rise above a threshold value and maintain the increase of p53 expression over a certain period of time in order to induce apoptosis in cancer cells by treatment with a platinum-based anticancer drug, that is, to effectively kill cancer cells by maintaining the increased expression of intracellular p53 by platinum-based anticancer drugs for a longer time. Then, the inventors of the present invention found that when a substance that inhibits ubiquitin ligase to p53, which is known as an intrinsic inhibitor against p53, was co-administered with cisplatin, the intracellular expression level of p53 increased by cisplatin maintained for a longer period of time and that its therapeutic effect on cancer cells could be better, and the present invention was completed.

Accordingly, an aspect of the present invention is to provide a method for maintaining elevated levels of p53 in cells, by administering a platinum-based anticancer drug and an siRNA against ubiquitin ligase to p53 to a subject in need thereof, in combination or sequentially.

Another aspect of the present invention is to provide a composition for promoting apoptosis by maintaining the elevated level of p53 in a cell, comprising, as an active ingredient, a platinum-based anticancer drug and siRNA against ubiquitin ligase to p53.

Another aspect of the present invention is to provide a pharmaceutical composition for preventing or treating cancer by maintaining elevated levels of p53 in cancer cells, comprising, as an active ingredient, a platinum-based anticancer drug and an siRNA against ubiquitin ligase to p53.

Another aspect of the present invention is to provide a method for screening preventive or therapeutic agents of cervical cancer, the method comprising steps (a) transducing a cervical cancer cell line with siRNA against E6 and E7 of Human Papillomavirus virus (HPV); (b) treating the cell line transduced in the step (a) with a cervical cancer therapeutic candidate substance; (c) measuring the expression level of intracellular p53 (tumor protein 53) at regular intervals for up to 48 hours from immediately after therapeutic candidate substance is treated; and (d) selecting a substance having a prolonged increase in expression level of intracellular p53 (tumor protein 53) as compared to a cell not treated with a therapeutic candidate substance.

### Technical Solution

In one embodiment, according to the present invention, the present invention provides a method for maintaining elevated levels of p53 in cells, by administering a platinum-based anticancer drug and an siRNA against ubiquitin ligase to p53 to a subject in need thereof, in combination or sequentially.

In another embodiment, according to the present invention, the present invention provides a composition, comprising, as an active ingredient, a platinum-based anticancer drug and siRNA against ubiquitin ligase to p53, for promoting apoptosis by maintaining the elevated level of p53 in a cancer cell.

In another embodiment, according to the present invention, the present invention provides a pharmaceutical composition for therapeutic use for maintaining elevated levels of p53 in cells, comprising, as an active ingredient, a platinum-based anticancer drug and an siRNA against ubiquitin ligase to p53.

In another embodiment, according to the present invention, the present invention provides a method for screening preventive or therapeutic agents of cervical cancer comprising steps (a) transducing a cervical cancer cell line with siRNA against E6 and E7 of Human Papillomavirus (HPV); (b) treating the cell line transduced in the step (a) with a cervical cancer therapeutic candidate substance; (c) measuring the expression level of intracellular p53 (tumor protein 53) at regular intervals for up to 48 hours from immediately after therapeutic candidate substance is treated; and (d) selecting a substance having a prolonged increase in expression level of intracellular p53 (tumor protein 53) as compared to a cell not treated with a therapeutic candidate substance.

Hereinafter, the present invention will be described in detail.

The present invention provides a method of maintaining elevated levels of p53 in cells, by administering a platinum-based anticancer drug and an siRNA against ubiquitin ligase to p53 to a subject in need thereof, in combination or sequentially.

In the present invention, the platinum-based anticancer drug is one of the anticancer drugs widely used in cancer treatment, and is used in about 50% of cancer patients. Platinum-based anticancer drugs are complexes that are coordinated with platinum. And in the present invention, the platinum-based anticancer drug may be selected from the group consisting of cisplatin (cis-diamminedichloroplatinum [II]), carboplatin, oxaliplatin, nedaplatin, picoplatin, triplatin tetranitrate, satraplatin, and mixtures thereof, preferably cisplatin or carboplatin, and most preferably cisplatin.

Among them, cisplatin is a compound having a structure represented by the following, Formula 1 in which two chlorine atoms and ammonia are coordinated to a platinum atom:

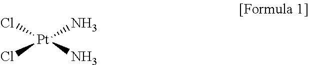

Unless otherwise stated, the platinum-based anticancer drug according to the present invention is used as a concept including both the compound itself, its pharmaceutically acceptable salts, hydrates, solvates, isomers and prodrugs thereof.

As used herein, the term “Pharmaceutically acceptable salt” means a formulation of a compound that does not cause serious irritation to the organism to which the compound is administered and does not impair the biological activity and properties of the compound. The terms “hydrate”, “solvate” and “isomer” also have the same meaning as above. The above pharmaceutical salts can be obtained by reacting the compounds of the present invention with Inorganic acids (such as hydrochloric acid, hydrobromic acid, sulfuric acid, nitric acid, and phosphoric acid), sulfonic acids (such as methanesulfonic acid, ethanesulfonic acid and p-toluenesulfonic acid), organic carboxylic acids (such as tartaric acid, formic acid, citric acid, acetic acid, trichloroacetic acid, trifluoroacetic acid, capric acid, isobutanoic acid, malonic acid, succinic acid, phthalic acid, gluconic acid, benzoic acid, lactic acid, fumaric acid, maleic acid, salicylic acid and the like). In addition, the above pharmaceutical salts of the present invention can be obtained by reacting the compound of the present invention with a base to form an alkali metal salt (such as an ammonium salt, sodium or potassium salt), an alkaline earth metal salt (such as calcium or magnesium salt), salts of organic bases (such as dicyclohexylamine, N-methyl-D-glucamine, tris (hydroxymethyl) methylamine), and amino acid salts such as salts of arginine, lysine and the like.

The “hydrate” means a compound of the present invention or a salt thereof, comprising a stoichiometric or non-stoichiometric amount of water bound by non-covalent intermolecular forces.

The “solvate” means a compound of the present invention or a salt thereof comprising a stoichiometric or non-stoichiometric amount of a solvent bound by noncovalent intermolecular forces. Preferred solvents therefor are volatile, non-toxic, and/or solvents suitable for administration to humans.

The “isomer” means a compound of the present invention or a salt thereof, which has the same chemical or molecular formula but is optically or sterically different. For example, the compound of Formula 1 according to the present invention may have an asymmetric center depending on the kinds of substituents. In this case, the compound of Formula 1 may exist as an optical isomer such as an enantiomer or a diastereomer.

The “prodrug” means a substance that is transformed into a parent drug in vivo. Prodrugs are often used in some cases because they are easier to administer than parent drugs. For example, they may obtain viability by oral administration, whereas parent drugs may not. Prodrugs may also have improved solubility in pharmaceutical compositions over the parent drug. For example, water solubility of prodrugs is detrimental to portability, but in cells that are once beneficial in water solubility, the prodrug will be a compound that is administered as an ester that facilitates passage of the cell membrane, which is hydrolyzed to the carboxylic acid that is active by metabolism. Another example of a prodrug may be a short peptide (polyamino acid) attached to an acid group that is converted by metabolism so that the peptide reveals its active site.

In the present invention, the ubiquitin ligase is a protein known as E3 ubiquitin ligase, and is a ubiquitin ligating enzyme that recognizes a specific protein and induces ubiquitination. The ubiquitin ligase acts as a ubiquitin-binding enzyme for p53 and functions to decompose p53 through ubiquitination and 26S proteasome pathway. Therefore, in the present invention, the ubiquitin ligase may be understood not only to include a protein which acts as a ubiquitin-binding enzyme for p53 to ubiquitinate p53, thereby decreasing the expression of p53 in cells, but also a fragment thereof, a functional equivalent thereof, and a functional derivative thereof. The ubiquitin ligase of the present invention includes two or more proteins that form a complex to function as ubiquitin ligase, but are not limited to, ubiquitin ligase complexes such as the E6/E6-AP complex of Human Papillomavirus (HPV).

The “fragment” means that although a portion of the amino acids of the native type protein is deleted, the physiological activity of the protein is the same as that of the native type protein or includes some of the constitutive proteins forming the protein complex. The “functional equivalent” is an amino acid sequence variant in which some or all of the native protein amino acids are substituted, or a part of the amino acids are deleted or added, and have physiological activity substantially equivalent to that of the native ubiquitin ligase protein.

In the present invention, the ubiquitin ligase may preferably be HDM2, E6/E6-AP complex of human Papillomavirus (HPV), E6 or E6-AP6 protein, and most preferably E6/E6-AP complex of human Papillomavirus (HPV), E6 or E6-AP protein, but is not limited thereto.

According to one example of the present invention, cisplatin treatment of cervical cancer cell lines revealed that the expression level of intracellular p53 was in the form of a pulse and repeatedly increased and decreased. In order for apoptosis of tumor cells to be induced by cisplatin, the expression level of intracellular p53 should be increased to a certain level (threshold value) or more. It was confirmed that p53 expression was not maintained to an extent sufficient to exhibit cytotoxicity, when tumor cells were treated with cisplatin alone.

According to another example of the present invention, the present inventors treated siRNA against E6 and E7 of Human Papillomavirus (HPV), while functions as a p53 ubiquitin ligase in cervical cancer cell lines, into cervical cancer cell lines, and the result also revealed that the expression level of p53 in the cells tended to increase, but the increase in the expression of p53 was not sustained for a long time enough to cause cytotoxicity.

Thus, the present inventors treated cervical cancer cell lines with siRNA against HPV E6 and E7 in combination with cisplatin. As a result, it was confirmed that the expression of p53 was maintained above the threshold level at which apoptosis of the cells could be induced.

The term “siRNA” in the present invention means a short double-stranded RNA capable of inducing RNAi (RNA interference) phenomenon through cleavage of a specific mRNA. The siRNA consists of a sense RNA strand having a sequence homologous to the mRNA of a target gene and an antisense RNA strand having a sequence complementary thereto. Since siRNA can inhibit the expression of a target gene, it is provided as an efficient method of gene knockdown or as a method of gene therapy.

siRNAs as used herein are not limited to the complete pairing of double-stranded RNA portions that pair with each other, and may include a non-paired portion due to a mismatch (the corresponding base is not complementary), a bulge (no base corresponding to one of the chains), and the like. The total length of the siRNA is 10 to 100 bases, preferably 15 to 80 bases, more preferably 20 to 70 bases. Both a blunt end and a cohesive end may be acceptable as a siRNA terminal structure as long as they can inhibit the expression of the target gene by RNAi effect. The structure of cohesive termini can have both 3-terminal protruding structure and 5-terminal protruding structure. The number of protruding bases is not limited. For example, the number of the protruding base may be from 1 to 8 bases, preferably from 2 to 6 bases. In the present specification, the total length of the siRNA is represented by the sum of the length of the central double-stranded portion and the length constituting the single-stranded protrusion at both ends. In addition, the siRNA may include, for example, a low molecular RNA (for example, a natural RNA molecule such as a tRNA, a rRNA, a viral RNA, or an artificial RNA molecule) at a protruding portion at one end in a range that can maintain the effect of inhibiting the expression of the target gene. The siRNA terminal structure does not have to contain a cleavage structure on both sides, and may be a stem loop type structure in which one terminal region of double-stranded RNA is connected by linker RNA. The length of the linker is not particularly limited as long as it does not interfere with the pairing of the stem portions.

The sequence and length of the siRNA in the present invention are not particularly limited as long as it can specifically reduce the ubiquitin ligase mRNA to p53. In the specific examples of the present invention, five kinds of siRNA specific to HPV E6 or E7 were prepared respectively, and it was confirmed that the siRNA increases the expression of intracellular p53 and has an effect of maintaining the expression of intracellular p53 at a threshold level or higher for a long time when the combination treatment with cisplatin is performed.

Therefore, in the present invention, the siRNA may be selected from the group consisting of SEQ ID NOS: 1 to 10, but is not particularly limited thereto.

The inventors of the present invention prepared siRNAs to E6/E7 tumorigenic proteins of HPV types 16 and 18 as shown in the following Table. In the following Table, the siRNA sequences indicated by ‘m’ represent a base substituted with a 2′-O-Me modified nucleotide in which a methyl group is bonded to a base residue. That is, in the case of 2′-O-Me modified U, it is indicated as ‘mU’, or in case of 2′-O-Me modified G, it is indicated as ‘mG’.

TABLE 1
siRNA to HPV types 16 and 18

| Name | Seq No. | Sequences |
|---|---|---|
| HPV type 18 | Seq No. 1 | 5′-CAACCmGAmGCACmGACAmGmGAA-3′ |
| siRNA 426 | (Forward) | |
| | Seq No. 2 | 5′-UUCCUGUCGUGCUCGGUUG-3′ |
| | (Reverse) | |
| HPV type 18 | Seq No. 3 | 5′-CCAACmGACmGCAmGAmGAAACA-3′ |
| siRNA 450 | (Forward) | |
| | Seq No. 4 | 5′-UGUmUUCUCmUGCGmUCGmUUGG-3′ |
| | (Reverse) | |
| HPV type 16 | Seq No. 5 | 5′-GCAAAGACAUCmUmGmGACAAA-3′ |
| siRNA 366 | (Forward) | |
| | Seq No. 6 | 5′-UUUGUCCAGAUGUCUUUGC-3′ |
| | (Reverse) | |
| HPV type 16 | Seq No. 7 | 5′-UCAAmGAACACmGUAmGAmGAAA-3′ |
| siRNA 488 | (Forward) | |
| | Seq No. 8 | 5′-UUUCUCUACGUGUUCUUGA-3′ |
| | (Reverse) | |
| HPV type 16 | Seq No. 9 | 5′-GACCGGUCGAUGUAUGUCUUG-3′ |
| siRNA 497 | (Forward) | |
| | Seq No. 10 | 5′-AGACAmUACAmUCGACCGGmUCCA-3′ |
| | (Reverse) | |

The present invention is characterized by the co-administration of platinum-based anticancer drug and a siRNA against ubiquitin ligase to p53 in order to maintain the increased expression of intracellular p53 by platinum-based anticancer drugs.

Such co-administration means that the platinum-based anticancer drug and the siRNA are administered simultaneously, separately or sequentially. More specifically, the platinum-based anticancer drug and the siRNA may be prepared in the form of a single composition and administered simultaneously, or one of the platinum-based anticancer drug and the siRNA is administered before the other one is administered. In the present invention, the order of administration of the platinum-based anticancer drug and the siRNA, that is, whether or not to administer the drugs simultaneously, separately or sequentially at a certain point in time, can be easily selected by the judgment of an expert.

When the platinum-based anticancer drug and the siRNA against ubiquitin ligase to p53 are co-administered to a subject according to the method of the present invention, the increased expression of p53 can be maintained for a long time in the cell of a subject, particularly cancer cells, resulting in induction of cancer cell death. Preferably, the death of the cancer cells may be through an apoptosis pathway induced by increased p53.

Another embodiment of the present invention provides a composition, comprising, as an active ingredient, a platinum-based anticancer drug and siRNA against ubiquitin ligase to p53, for promoting apoptosis by maintaining the elevated level of p53 in a cancer cell.

Another embodiment of the present invention provides a pharmaceutical composition for therapeutic use for maintaining elevated levels of p53 in cells, comprising, as an active ingredient, a platinum-based anticancer drug and an siRNA against ubiquitin ligase to p53.

The pharmaceutical composition according to the present invention can be formulated in the form of a single composition or in the form of a separate composition. Preferably, they can be formulated in the form of individual compositions. Methods for formulating them can utilize a technique commonly used in the art.

In the pharmaceutical composition according to the present invention, each of the above components may be administered simultaneously, separately or sequentially. For example, when each component contained in the pharmaceutical composition of the present invention is a single composition, it may be administered at the same time. In the case that the composition is not a single composition, one component may be administered while the other component is administered before, after, and/or with other components. The order of administration of the pharmaceutical composition according to the present invention, i.e., the timing of administration and simultaneous, separate or sequential administration of the pharmaceutical composition according to the present invention may be determined by a doctor or an expert. The order of administration may vary depend on many factors.

According to one example of the present invention, when cisplatin and siRNA against HPV E6 and E7 were co-administered to an animal model transplanted with human cervical cancer cells, it was found that excellent tumor growth inhibitory effect was exhibited even at a low concentration at which no tumor growth inhibitory effect was induced by administration of cisplatin alone.

That is, the co-administration of the platinum-based anticancer drug and the siRNA using the pharmaceutical composition of the present invention could advantageously reduce serious side effects, which can be caused by excessive or long-term administration of platinum-based anticancer drugs, can be reduced because of its excellent anticancer effect even when the drug is administered at a lower concentration than the concentration of the platinum-based anticancer drug conventionally administered for the anticancer effect.

Accordingly, the pharmaceutical composition of the present invention can exhibit an excellent anticancer effect by maintaining an increased expression of p53 in cancer cells for a long time, and can also be usefully applied to clinics because it can alleviate adverse effects due to chemotherapy.

The pharmaceutical composition according to the present invention may be formulated into a suitable form together with the platinum-based anticancer drug and the siRNA themselves or in combination with a pharmaceutically acceptable carrier, and may further contain an excipient or a diluent. Such carriers include all types of solvents, dispersion media, oil-in-water or water-in-oil emulsions, aqueous compositions, liposomes, microbeads and microsomes.

The pharmaceutically acceptable carrier may further include, for example, a carrier for oral administration or a carrier for parenteral administration. Carriers for oral administration may include lactose, starch, cellulose derivatives, magnesium stearate, stearic acid, and the like. In addition, it may contain various drug delivery materials used for oral administration. In addition, the carrier for parenteral administration may contain water, a suitable oil, a saline solution, an aqueous glucose and a glycol, and may further contain a stabilizer and a preservative. Suitable stabilizers include antioxidants such as sodium hydrogen sulfite, sodium sulfite or ascorbic acid. Suitable preservatives include benzalkonium chloride, methyl- or propyl-paraben and chlorobutanol. The pharmaceutical composition of the present invention may further contain a lubricant, a wetting agent, a sweetener, a flavoring agent, an emulsifying agent, a suspending agent and the like in addition to the above components. Other pharmaceutically acceptable carriers and preparations can be referred to those described in the following references (Remington's Pharmaceutical Sciences, 19th ed., Mack Publishing Company, Easton, Pa., 1995).

The composition of the present invention can be administered to mammals including humans by any method. For example, it can be administered orally or parenterally. Parenteral administration methods include, but are not limited to, intravenous, intramuscular, intraarterial, intramedullary, intrathecal, intracardiac, transdermal, subcutaneous, intraperitoneal, intranasal, enteral, topical, sublingual or rectal administration.

The pharmaceutical composition of the present invention may be formulated into oral or parenteral administration preparations according to the administration route as described above.

In the case of oral administration preparations, the composition of the present invention may be formulated into powder, granules, tablets, pills, sugar tablets, capsules, liquids, gels, syrups, slurries, suspensions or the like using methods known in the art. For example, an oral preparation can be obtained as tablets or sugar tablets by combining the active ingredient with a solid excipient, then milling it, adding suitable auxiliaries, and processing the mixture into granules. Examples of suitable excipients include sugars including lactose, dextrose, sucrose, sorbitol, mannitol, xylitol, erythritol and maltitol and the like, and starches including corn starch, wheat starch, rice starch and potato starch, cellulose such as cellulose, methyl cellulose, sodium carboxymethyl cellulose and hydroxypropylmethyl cellulose and the like, fillers such as gelatin, polyvinylpyrrolidone and the like. In addition, crosslinked polyvinylpyrrolidone, agar, alginic acid or sodium alginate may optionally be added as a disintegrant. Further, the pharmaceutical composition of the present invention may further comprise an anti-coagulant, a lubricant, a wetting agent, a flavoring agent, an emulsifying agent and an antiseptic agent.

The preparation for parenteral administration may be formulated in the form of injections, creams, lotions, ointments, oils, moisturizers, gels, aerosols and nasal inhalers by methods known in the art. These formulations are described in the literature (Remington's Pharmaceutical Science, 19th ed., Mack Publishing Company, Easton, Pa., 1995), which is a prescription manual commonly known in all pharmaceutical chemistries.

The total effective amount of the composition of the present invention may be administered to a patient in a single dose and may be administered by a fractionated treatment protocol administered over a prolonged period of time in multiple doses. The dosage of the pharmaceutical composition may be determined depending on various factors such as the formulation method, administration route, and the number of treatments as well as the patient's age, weight, health condition, sex, severity of disease, diet and excretion rate. With this in mind, one of ordinary skill in the art will be able to determine the appropriate effective dose of the composition of the present invention. According to Ministry of Food and Drug Safety (KFDA) data, platinum-based anticancer drugs can be administered in doses of 0.1 to 250 μM per each administration.

The effective amount of the platinum-based anticancer drug is preferably 0.1 to 250 μM, more preferably 0.5 to 200 μM, most preferably 0.625 to 160 μM, but is not limited thereto, and the effective amount may be adjusted by a doctor depending on the patient's age, height, severity, site and excretion amount of the patient.

On the other hand, when the pharmaceutical composition containing the platinum-based anticancer drug and the siRNA is administered in the form of a single preparation, a preparation is appropriately selected so that the dosage of the platinum-based anticancer drug and the siRNA is one or less of the amount of the platinum-based anticancer drug, and the compounding agent can be administered once or several times a day. Preferably, the platinum-based anticancer drug is administered at a dose of 0.5 to 200 μM per administration, and the siRNA for ubiquitin ligase to p53 is administered at a dose of 0.5 mg to 5 mg per day, and these doses can be administered in several divided doses.

The effective amount of the siRNA for ubiquitin ligase to p53 is preferably 0.1 to 20 mg/kg, more preferably 0.2 to 15 mg/kg, most preferably 0.4 to 10 mg/kg, but is not limited thereto, and the effective amount may be adjusted by a doctor depending on the patient's age, height, severity, site and excretion amount of the patient.

The pharmaceutical composition according to the present invention is not particularly limited to the formulation, administration route and administration method as long as the effect of the present invention is exhibited.

One embodiment of the present invention provides a method for screening preventive or therapeutic agents of cervical cancer comprising steps (a) transducing a cervical cancer cell line with siRNA against E6 and E7 of Human Papillomavirus (HPV); (b) treating the cell line transduced in the step (a) with a cervical cancer therapeutic candidate substance; (c) measuring the expression level of intracellular p53 at regular intervals for up to 48 hours from immediately after therapeutic candidate substance is treated; and (d) selecting a substance having a prolonged increase in expression level of intracellular p53 as compared to a cell not treated with a therapeutic candidate substance.

The method of transfecting cervical cancer cell lines with siRNA against HPV E6 and E7 in the step (a) of the present invention includes transfection with calcium phosphate (Graham, F L et al., Virology, 52: 456 (1973)), transfection with DEAE dextran, transfection by microinjection (Capecchi, M R, Cell, 22: 479 (1980)), transfection by cationic lipids (Wong, T K et al., Gene, 10:87 1980), electroporation (Neumann E. et al., EMBO J., 1: 841 (1982)), transduction or transfection, and the like, which are well known to those skilled in the art can be prepared according to methods as described in the literature (Basic methods in molecular biology, Davis et al., 1986, Molecular cloning: A laboratory manual, Davis et al., 1986).

In the step (b) of the present invention, the candidate therapeutic substance means an unknown substance which is expected to affect the expression amount of p53 in a cell, and includes antibodies, aptamers, natural extracts or chemical substances, but is not limited thereto. Treatment of a candidate therapeutic agent with a cell means that the cell is incubated for a certain period of time after adding the test substance to the cell culture medium.

The expression of p53 in the step (c) means the expression of p53 mRNA or protein, which can be measured by a method selected from the group consisting of RT-PCR, competitive RT-PCR, real-time RT-PCR, RNase protection assay (RPA), Northern blotting, DNA microarray chip analysis, Western blotting, enzyme-linked immunosorbent assay, radioimmunoassay (RIA), radioimmunodiffusion, Ouchterlony immunodiffusion, rocket immunoelectrophoresis, Immunohistochemistry, immunoprecipitation, complement fixation, flow cytometry (FACS) and protein chip analysis.

The regular intervals in the step (c) means a time interval suitable for observing changes in the expression amount of p53 in the cells over time, and is not particularly limited, but preferably may be 30 minutes to 1 hour.

The screening method of the present invention may further include the step (e) after the step (d), the step of identifying the effect of the selected substance in a cell or an animal. Identifying its effect in the cells means to confirm whether it has a cytotoxicity to the cervical cancer cell line, while identifying its effect in the animal means to confirm whether it shows a tumor growth inhibitory effect in a tumor animal model transplanted with a cervical cancer cell line.

### Advantageous Effects

According to the method of the present invention, which is characterized by administering a platinum-based anticancer drug and an siRNA against ubiquitin ligase to p53 to a subject in need thereof, in combination or sequentially, it is possible to maintain the increased expression level of intracellular p53 for a long time even after treatment with a low dose of platinum-based anticancer drug, thereby effectively inducing the death of cancer cells and minimizing the side effects of drug administration upon administration of a platinum-based anticancer drug, so that it can be usefully used for the development of a preventive or therapeutic agent for cancer.

### BRIEF DESCRIPTION OF THE DRAWINGS

FIG. 1. is a graph showing the intracellular TP53 kinetics when cervical cancer cells were treated with HPV E6/E7 siRNA in combination with cisplatin (426, 450, 366, 448, 497: siRNA code name, SP: siRNA pool, CDDP: cisplatin, NC: no treatment control group).

FIG. 1A shows that Hela cells (left panel) and CaSki cells (right panel) were transduced with 20 nM of HPV E6/E7 siRNA and exposed to 10 μM of cisplatin. After 24 hours, whole cell lysates were collected for induction of TP53 and immunoblot analysis of E6, E7 efficiency. β-actin was used as a control.

FIG. 1B shows that cell viability was confirmed by WST analysis. The error bars mean the mean±SD of independent experiments.

FIG. 1C shows that the degree of induction of TP53 and hyperphosphorylated RB in tumor cells treated with HPV E6/E7 siRNA was evaluated using TP53 and E2F luciferase reporter activity.

FIG. 1D shows that Hela cells (upper panel) and CaSki cells (lower panel) were treated with E6/E7 specific siRNA alone and/or with cisplatin (CDDP). Silent restoration of endogenous TP53, RB and 18E7, 16E7, 16E6 and 18E6 was analyzed with other sub-target genes at designated time points. β-actin was used as a control.

FIG. 1E shows the ratio of the relative intensities of TP53, E6, E2F, and E7 obtained from corresponding western blots.

FIG. 1F shows that after treatment with Hela cells (left panel) and CaSki cells (right panel) with E6/E7 specific siRNA alone and/or CDDP, CDKN1A transcript levels in each cell were analyzed by real-time quantitative PCR (qPCR). The error bars mean the mean±SD of independent experiments.

FIG. 2 shows the results of real-time qPCR analysis of the effect of HPV E6/E7 siRNA alone or in combination with CDDP on the expression of a gene well known as a target gene of TP53 (A: Hela cells, B: CaSki cells).

FIG. 3 shows the results of analysis of the effect of E6/E7 deletion on TP53 kinetics and cell survival.

FIG. 3A shows that a schematic representation of the GFP-TP53 reporter construct was used to form stable cell lines. The characterized TP53-RE contains the TP53 consensus binding site (green box) and the arrows represent the minimal TATA box with the GFP-reporter gene. IncuCyte was used as 9 images of all wells every 30 minutes. Time-lapse microscopic images of live Hela cells were performed with 20 nM of SP, 10 μM of CDDP monotherapy, and mixed treatment of SP and CDDP, respectively. The selection of representative images after post-transduction was expressed by a fluorescence and phase-contrast image merge with a control siRNA-treated cell as a control group (Representative IncuCyte images of live cell video recordings made for 5 days. scale bar=40 μm. GFP-TP53 Reporter stable Hela cells).

FIG. 3B shows that growth rate (upper left), GFP count (upper right), GFP intensity (lower right), and the normalization of GFP counts (lower left) indicate CDDP alone and/or combination therapies in response to HPV E6/E7 siRNA (Scale bar: 20 μm. Arrows indicate induction of TP53-responsive element (RE)-driven GFP reporter gene expression and HPV E6/E7 siRNA treatment time).

FIG. 3C shows that growth rate (upper left), GFP count (upper right), GFP intensity (lower left), and GFP count normalization (lower right) of GFP-TP53 reporter stable CaSki cells in response to HPV E6/E7 siRNA alone and/or combination treatment (Arrows indicate induction of TP53-responsive element (RE) driven GFP reporter gene expression and HPV E6/E7 siRNA treatment time).

FIG. 3D shows a simulation of TP53 activity by HPV E6/E7 siRNA alone, CDDP alone or a combination thereof.

FIG. 4 shows the results of observing the expression of GFP-TP53 and RFP-E2F for HPV E6/E7 siRNA alone and/or in combination with CDDP.

FIG. 4A shows that stable Hela cells transformed with cyanene RFP-E2F and GFP-TP53 were cultured and time-dependent changes of RFP-E2F in GFP-TP53 were photographed by confocal microscopy to demonstrate the silencing effect of the HPV E6/E7 oncogene in the Hela cells (red indicates Ex/Em=565 nm/650 nm and green indicates Ex/Em=495 nm/545 nm). The fluorescence image of the cell overlaps the phase difference image).

FIG. 4B shows that mean intensity data were obtained as a time-lapse confocal image of Dual reporter stable cells. Cells were taken every 20 minutes from 12 hours to 24 hours. In particular, the metastatic pattern was observed in HPV E6/E7 siRNA pool transformed cells at 19 to 21 hours

FIG. 5 shows the results that the effect of triple combination treatment (cisplatin, paclitaxel and siRNA) on HPV positive cervical cancer cells was confirmed in vitro or in vivo (CDDP: cisplatin, PTX: paclitaxel).

FIG. 5A shows that Hela cells were treated with E6/E7 specific siRNA, CDDP+paclitaxel, or a mixture thereof. The induction of endogenous TP53, hypophosphorylated RB and silencing 18E7, 16E7, 16E6 and 18E6 were analyzed with other sub-target genes at the indicated time points. β-actin was used as a control.

FIG. 5B shows that CDKN1A transcript levels were analyzed by real-time quantitative (q) PCR. The error bars represent the mean±SD of independent experiments.

FIG. 5C shows that the effects of the three mixtures on the activity of GFP-TP53 were analyzed and exhibited as cell growth rate (upper left), GFP count (upper right), and GFP intensity (lower right).

FIG. 5D shows that Hela cells were treated with HPV E6/E7 siRNA pools, CDDP and CDDP+paclitaxel, or mixtures thereof. Cell cycle analysis and percentage of cells in each step (G0-G1, S and G2/M) were measured.

FIG. 6 shows a graphical representation of the role of HPV E6/E7 oncogene and HPV E6/E7 siRNA combination therapy in HPV-infected cervical cancer cells.

FIG. 7 shows the results of checking the expression level of GFP-TP53 over time in HeLa cells when cisplatin alone, siRNA pool alone and/or cisplatin were treated.

### DETAILED DESCRIPTION OF THE INVENTION

Hereinafter, the present invention will be described in detail.

However, the following examples are illustrative of the present invention, and the present invention is not limited to the following examples.

### Example 1

Effect of Cisplatin and HPV E6/E7 siRNA on TP53 in Cervical Cancer Cells

### Example 1-1

Effect of HPV E6/E7 siRNA co-administered with Cisplatin in Cervical Cancer Cells

Cervical cancer cell and HeLa cervical cancer cell lines (HeLa; ATCC CCL-2) infected with HPV type 18 virus and SiHa cervical cancer cell lines (SiHa; ATCC HTB-35) infected with HPV type 16 virus were plated on a 6-well plate with 5×10⁴ or 1×10⁵ cells and cultured in RPMI 1640 or DMEM medium for 24 hours at 37° C. and 5% carbon dioxide, respectively. siRNA intracellular transfection was performed with DharmaFect (Dharmacon, Lafayette, Colo., USA) according to manufacturer's instructions.

HeLa cervical carcinoma cells infected with HPV type 18 virus were transformed by pooling with each of siRNAs of 426 and 450 sequences, or respectively. The HPV type 16 virus-infected siHa cell line was transformed by pooling siRNAs of 366, 448 and 497 sequences, or respectively.

In the Western blot, whole-cell lysate was extracted with RIPA buffer (10 mM Tris (pH.4), 0.15 M NaCl, 5 mM EDTA, 1% Triton X-100, 0.5% deoxycholic acid sodium salt and 0.1% SDS). After centrifugation, the protein concentration of the supernatant was measured using a BCA protein assay kit (Thermo Fisher Scientific Inc). Protein samples were placed in sample buffer and boiled for 5 minutes for complete denaturation. The samples were then applied to 6%, 10%, or 15% polyacrylamide gels and transferred to a 0.4 μm PVDF membrane (Milipore, Bilerica). After transfer, the membranes were blocked with 5% skim milk, incubated with the appropriate concentration of primary antibody and horseradish peroxidase-conjugated IgG. Finally, the expression level of the protein was detected using ECL solution. The antibody used in the Western blotting experiment was TP53 [DO7], HPV 18-E6 [G7], HPV16-E6 [C1P5], HPV 18-E7 [F7], HPV16-E7 [ED17], while antibodies from Santa Cruz Biotechnology (St. Louis, Calif.) were used for 13-Actin [C4].

To investigate the effect of HPV E6/E7 siRNA conjugated with cisplatin (CDDP) on TP53 in cervical cancer cells, the following experiment was conducted.

Hela cells and CaSki cells, which are cervical cancer cells, were transformed with HPV E6/E7 siRNA and treated with 10 μM of cisplatin for 24 hours. Cell lysates were collected and subjected to western blotting by methods known in the art.

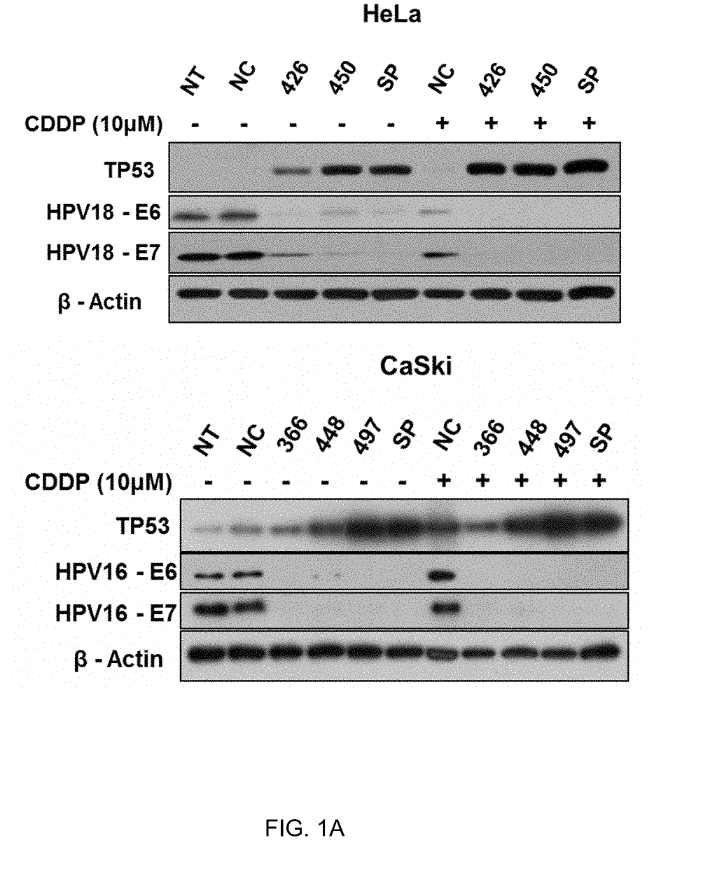

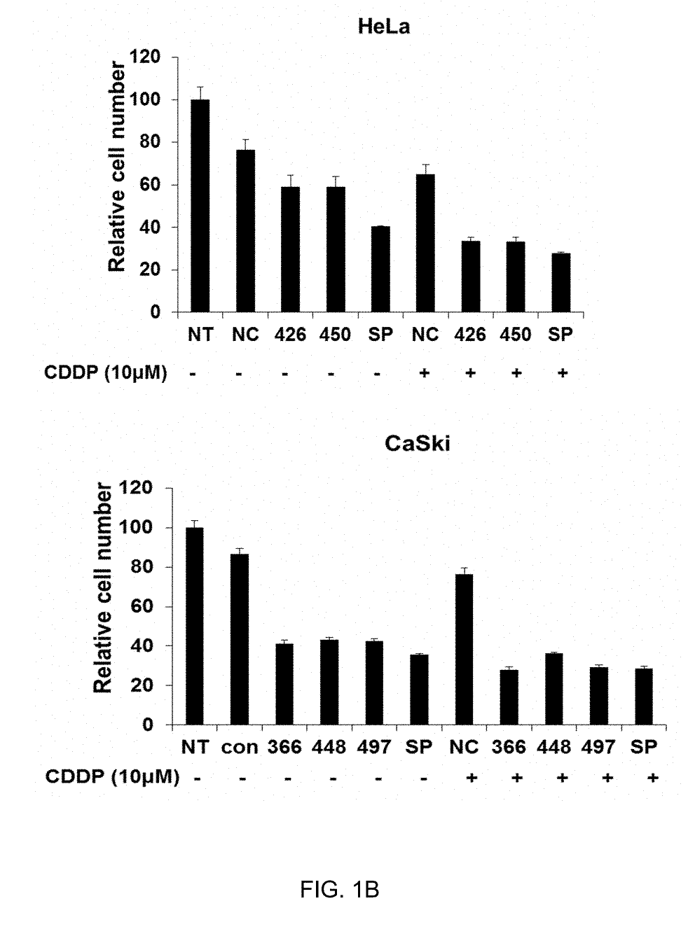

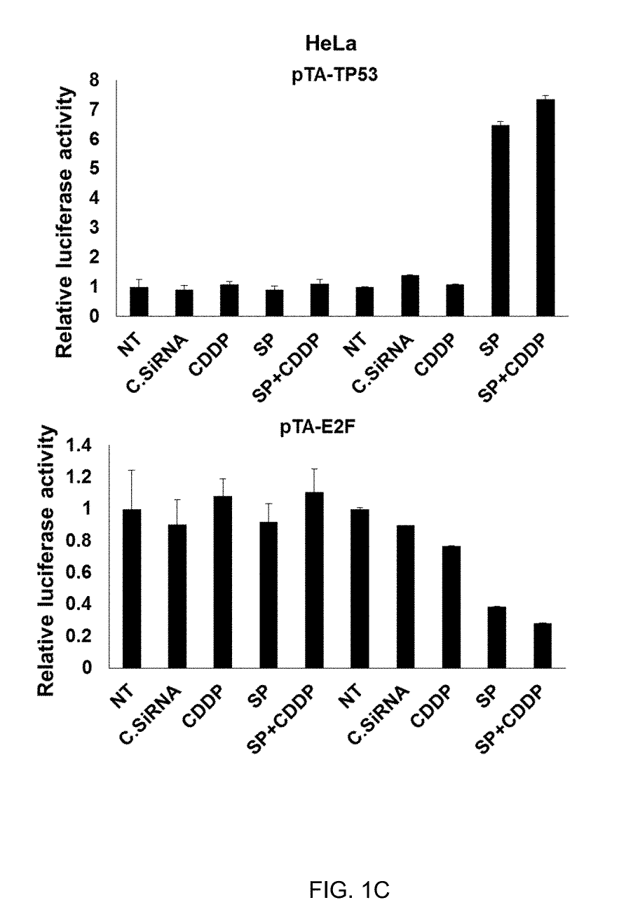

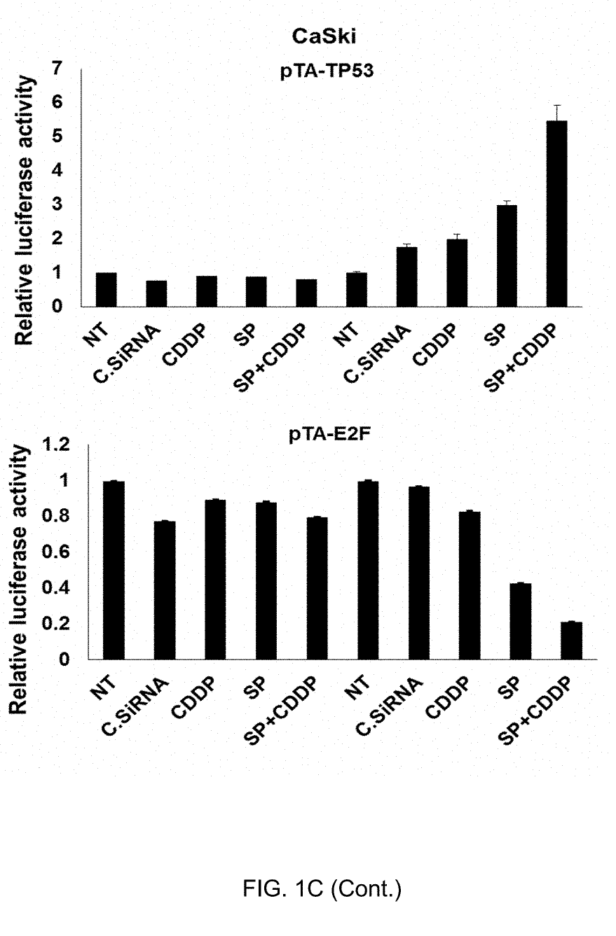

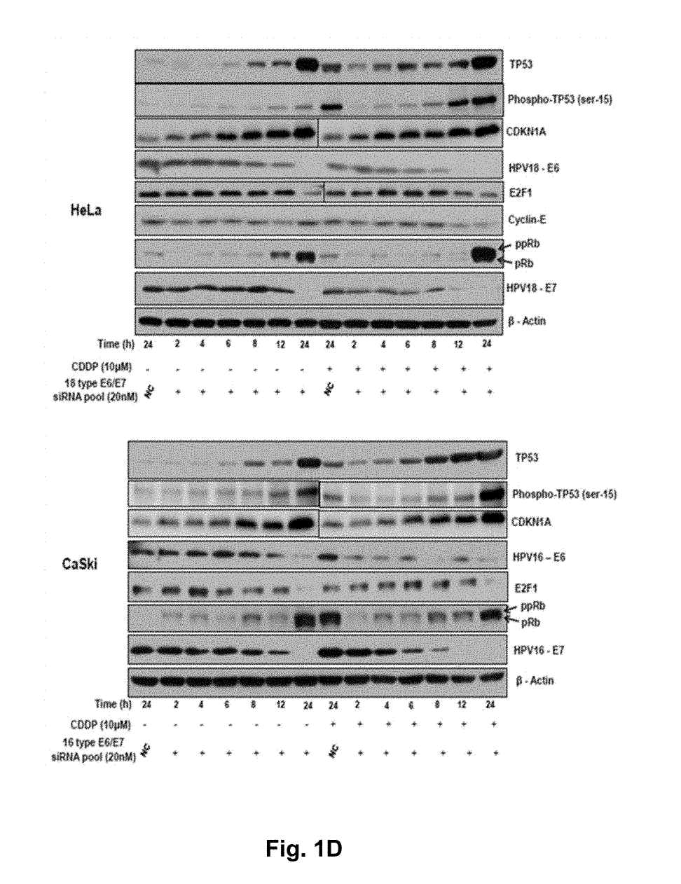

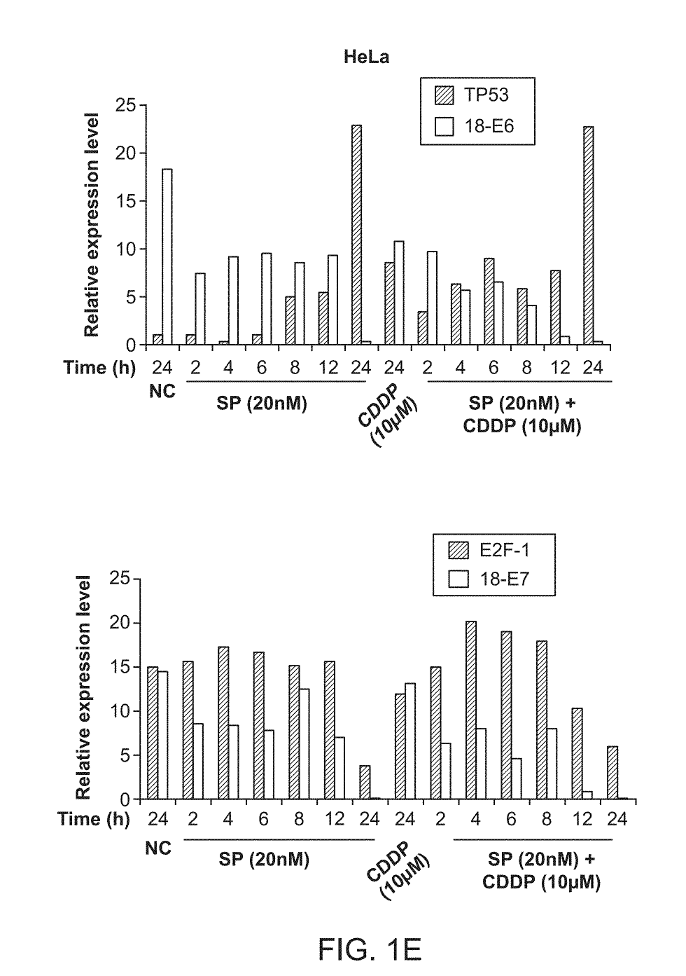

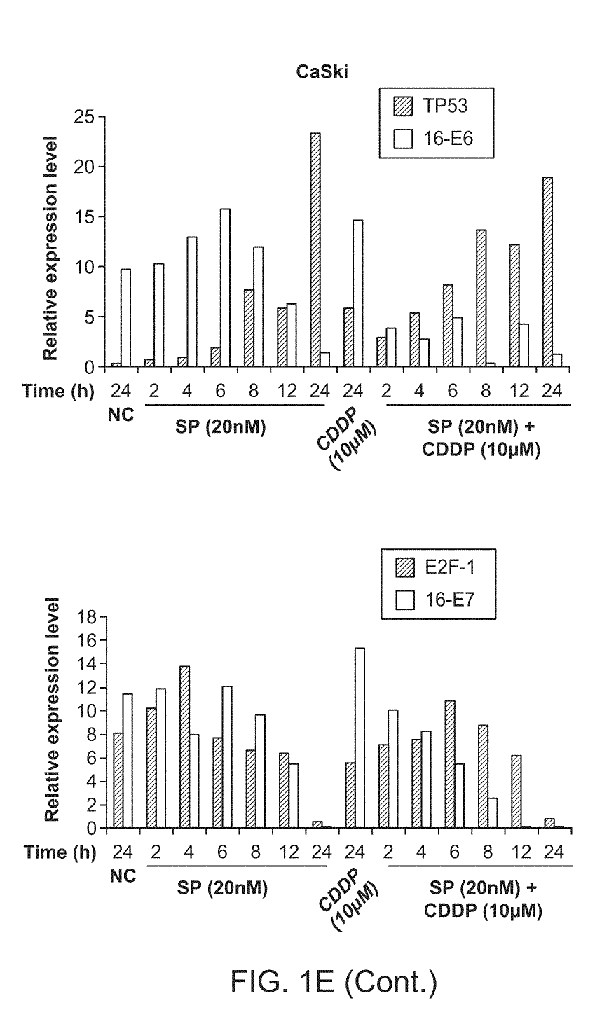

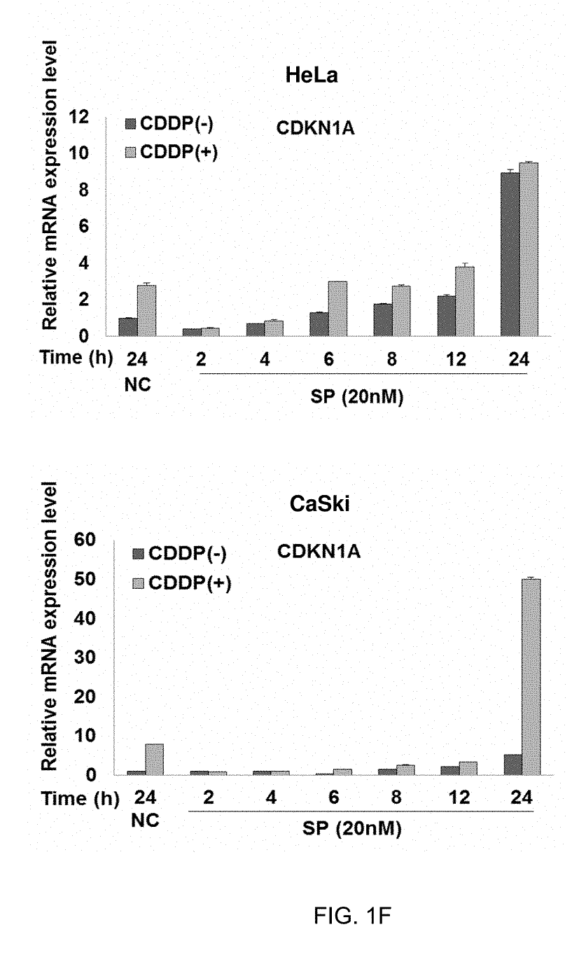

As a result, as shown in FIG. 1A, the amount of HPV E6/E7 protein expression was not observed in the group treated with cisplatin, and the amount of TP53 expression was increased.

### Example 1-2

Cell Apoptotic Effects of Cisplatin and HPV E6/E7 siRNA

To investigate the effect of cisplatin (CDDP) and HPV E6/E7 siRNA on cell viability in cervical cancer cells, the following experiment was conducted.

Cervical cancer cells, Hela and CaSki cells were transformed with HPV E6/E7 siRNA and cultured for 24 hours with 10 μM of cisplatin. WST analysis was performed to confirm cell viability.

Cell counts were determined by water soluble tetrazolium salts (WST) (EZ-Cytox kit; Daeil Lab Service, Seoul, Korea). EZ-Cytox solution (50 μl) added to each well of a 12-well plate and incubated for 2 to 3 hours. Live cells were measured (485 nm) using a GENios Pro microplate reader (Tecan Trading AG, Mannedorf, Switzerland) in a 96-well plate.

As a result, as shown in FIG. 1B, cell viability was decreased in the treated group compared to the group not treated with cisplatin

### Example 1-3

Measurement of enzyme activity of TP53 by Cisplatin and HPV E6/E7 siRNA

To investigate the effects of cisplatin (CDDP) and HPV E6/E7 siRNA on enzyme activity in cervical cancer cells, the following experiment was conducted.

Hela cells and CaSki cells, treated with cisplatin (CDDP) and HPV E6/E7 siRNA, were double-transformed with pTA-TP53-Luc (TP53 reporter) and pTA-E2F-Luc (E2F reporter) vectors. The above luciferase reporter vector system was purchased from Clontech pathway profiling system (Mountain View, Calif., USA). Luciferase assay was performed using Dual-Luciferase Reporter Assay System (Promega) after 24 hours of transfection, and luminescence activity was measured using a GENios Pro microplate reader (Tecan Trading AG, Mannedorf, Switzerland).

As a result, as shown in FIG. 1C, the cisplatin treatment of Hela cells and CaSki cells showed an increase in the activity of TP53, but a further decrease in the activity of E2F.

### Example 1-4

Effect of Cisplatin and HPV E6/E7 siRNA on the Expression of TP53

To investigate the effects of cisplatin (CDDP) and HPV E6/E7 siRNA on enzyme activity in cervical cancer cells, the following experiment was conducted.

Hela cells and CaSki cells were cultured and transformed in the same manner as described in Example 1-1, then treated with cisplatin. Cell lysates were then obtained and subjected to western blotting.

In western blotting, antibodies of Santa Cruz Biotechnology (St. Louis, Calif.) were used for TP53 [DO7], HPV 18-E6 [G7], HPV 16-E6 [C1P5], HPV 18-E7 [F7], HPV 16-E7 [ED17], E2F-1 [KH195], Cyclin-E [M20], β-Actin [C4] and antibody of BD Pharmingen was used for RB [554164], antibody of cell signaling was used for phospho-TP53 (ser-15) [9284].

As a result, as shown in FIGS. 1D and 1E, when cisplatin was treated, the expression level of TP53 was increased and the expression level of pRB was increased. In addition, the expression levels of 18-E6 and 16-E6, 18-E7 and 16-E7 were decreased (FIG. 1D, FIG. 1E).

### Example 1-5

Effect of Cisplatin and HPV E6/E7 siRNA on the Expression of CDKN1A

To investigate the effects of cisplatin (CDDP) and HPV E6/E7 siRNA on enzyme activity in cervical cancer cells, the following experiment was conducted.

Hela cells and CaSki cells were cultured and transformed in the same manner as described in the above example, then treated with cisplatin. RNA was then extracted from the cells and subjected to real-time qPCR.

The sequences of primers and probes used in the PCR were as follows and all were made in TIB MOLBIOL (Berlin, Germany).

CDKN1A forward
5′-CGA AGT CAG TTC CTT GTG GAG-3′
CDKN1A reverse
5′-CAT GGG TTC TGA CGG ACA T-3′
TaqMan probe
5′-FAM-CAG AGG AG-Dark quencher-3′

mRNA levels were determined at 530 and 705 nm wavelengths using the LightCycler Real-time PCR Detection System (Roche Diagnostics, Basel, Switzerland).

As a result, as shown in FIG. 1F, the amount of CDKN1A mRNA expression was increased when Hela cells (left) and CaSki cells (right) were treated with cisplatin and E6/E7 siRNA.

### Example 2

Effect of Cisplatin and HPV E6/E7 siRNA on the Expression of TP53-Related Genes in Cervical Cancer Cells

To investigate the effects of cisplatin (CDDP) and HPV E6/E7 siRNA on the expression of TP53 target genes in cervical cancer cells, the following experiment was conducted.

The well-known TP53 target genes were classified according to function by cell cycle arrest and DNA recovery, TP53 expression control, apoptosis and regulation of senescence. Hela cells and CaSki cells were cultured and transformed in the same manner as described in the above example, and treated with cisplatin. RNA was then extracted from the cells and subjected to real-time qPCR.

The sequences of primers and probes used in the PCR were as shown in below Table 2 and were all produced in TIB MOLBIOL (Berlin, Germany). mRNA levels were determined at 530 and 705 nm wavelengths using the LightCycler Real-time PCR Detection System (Roche Diagnostics, Basel, Switzerland).

TABLE 2
Sequences of primers and probes for qRT-PCR

| Name | Sequence |
|---|---|
| CDKN1A forward (Seq No. 11) | 5′-CGA AGT CAG TTC CTT GTG GAG-3′ |
| CDKN1A reverse (Seq No. 12) | 5′-CAT GGG TTC TGA CGG ACA T-3′ |
| TaqMan probe (Seq No. 13) | 5′-FAM-CAG AGG AG-Dark quencher-3′ |
| APAF1 forward (Seq No. 14) | 5′-CCT GTT GTC TCT TCT TCC AGT GT-3′ |
| APAF1 reverse (Seq No. 15) | 5′-AAA ACA ACT GGC CTC TGT GG-3′ |
| TaqMan probe (Seq No. 16) | 5′-FAM-AGG TGG AG-Dark quencher-3′ |
| BAX forward (Seq No. 17) | 5′-GAA CCA TCA TGG GCT GGA-3′ |
| BAX reverse (Seq No. 18) | 5′-CGT CCC AAA GTA GGA GAG GA-3′ |
| TaqMan probe (Seq No. 19) | 5′-FAM-CTT CCT CC-Dark quencher-3′ |
| PML forward (Seq No. 20) | 5′-GAG CCC CGT CAT AGG AAG T-3′ |
| PML reverse (Seq No. 21) | 5′-CAC AAC GCG TTC CTC TCC-3′ |
| TaqMan probe (Seq No. 22) | 5′-FAM-GCAGGAAG-Dark quencher-3′ |
| YPEL3 forward (Seq No. 23) | 5′-AAC CAC GAC GAC CTC ATC TC-3′ |
| YPEL3 reverse (Seq No. 24) | 5′-AGC CCA CGT TCA CCA CTG-3′ |
| TaqMan probe (Seq No. 25) | 5′-FAM-CCAGGGCA-Dark quencher-3′ |
| GADD45A forward (Seq No. 26) | 5′-CCC CGA TAA CGT GGT GTT-3′ |
| GADD45A reverse (Seq No. 27) | 5′-GCC ACA TCT CTG TCG TCG T-3′ |
| TaqMan probe (Seq No. 28) | 5′-FAM-GCC TGC TG-Dark quencher-3′ |
| XPC forward (Seq No. 29) | 5′-AGA CCA TAC CAG AGC CCA TTT-3′ |
| XPC reverse (Seq No. 30) | 5′-AGG CTG GTC CAT GTG TTT TG-3′ |
| TaqMan probe (Seq No. 31) | 5′-FAM-GGG AGA AG-Dark quencher-3′ |
| PPM1D (Wip1) forward (Seq No. 32) | 5′-CCC ATG TTC TAC ACC ACC AGT-3′ |
| PPM1D (Wip1) reverse (Seq No. 33) | 5′-TGG TCC TTA GAA TTC ACC CTT G-3′ |
| TaqMan probe (Seq No. 34) | 5′-FAM-TGG AGG AG-Dark quencher-3′ |
| MDM2 forward (Seq No. 35) | 5′-CCA TGA TCT ACA GGA ACT TGG TAG TA-3′ |
| MDM2 reverse (Seq No. 36) | 5′-TCA CTC ACA GAT GTA CCT GAG TCC-3′ |
| TaqMan probe (Seq No. 37) | 5′-FAM-TCC TGC TG-Dark quencher-3′ |
| HPRT1 forward (Seq No. 38) | 5′-TGA CCT TGA TTT ATT TTG CAT ACC-3′ |
| HPRT1 reverse (Seq No. 39) | 5′-CGA GCA AGA CGT TCA GTC CT-3′ |
| TaqMan probe (Seq No. 40) | 5′-FAM-GCTGAGGA-Dark quencher-3′ |

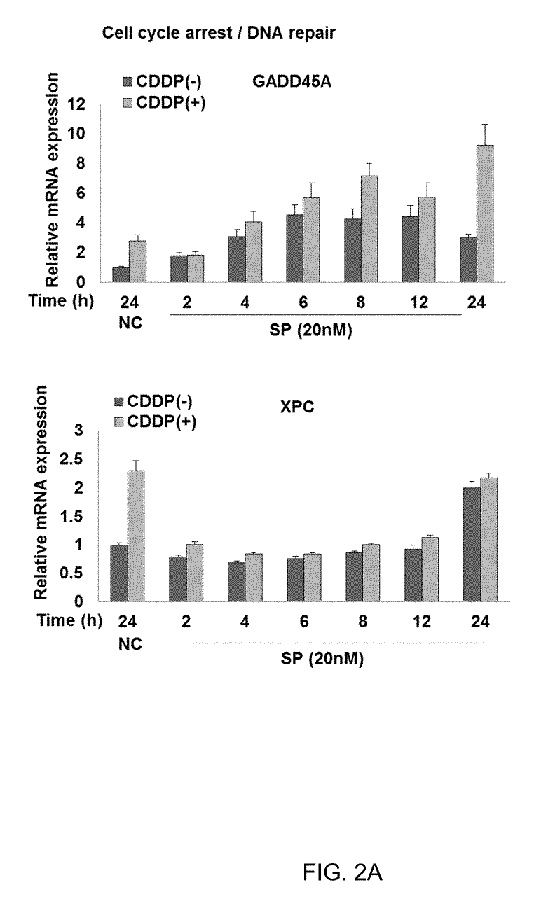

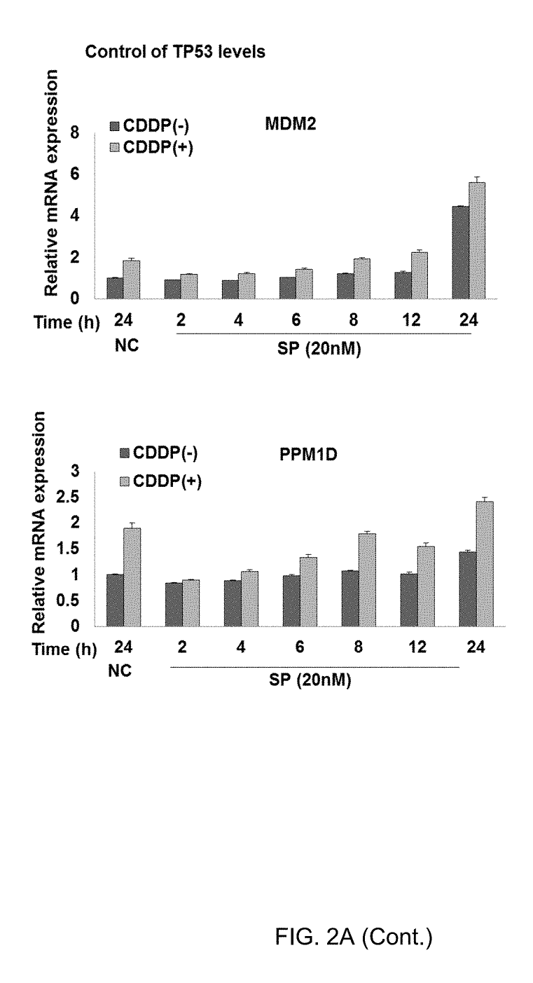

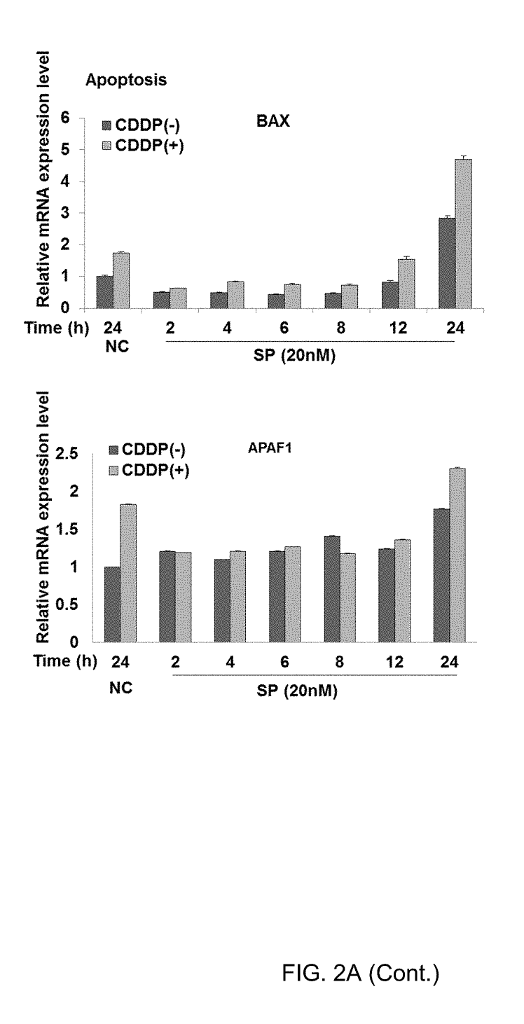

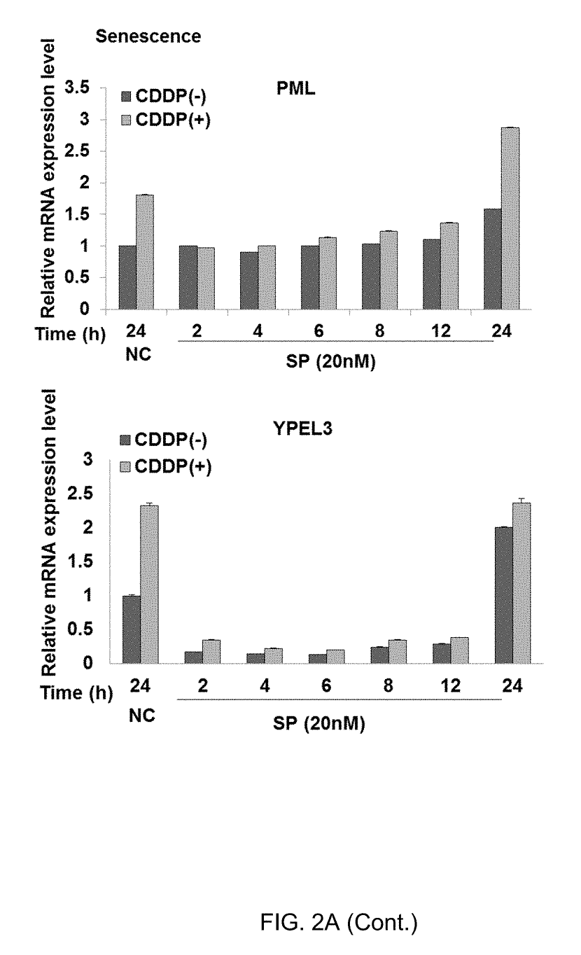

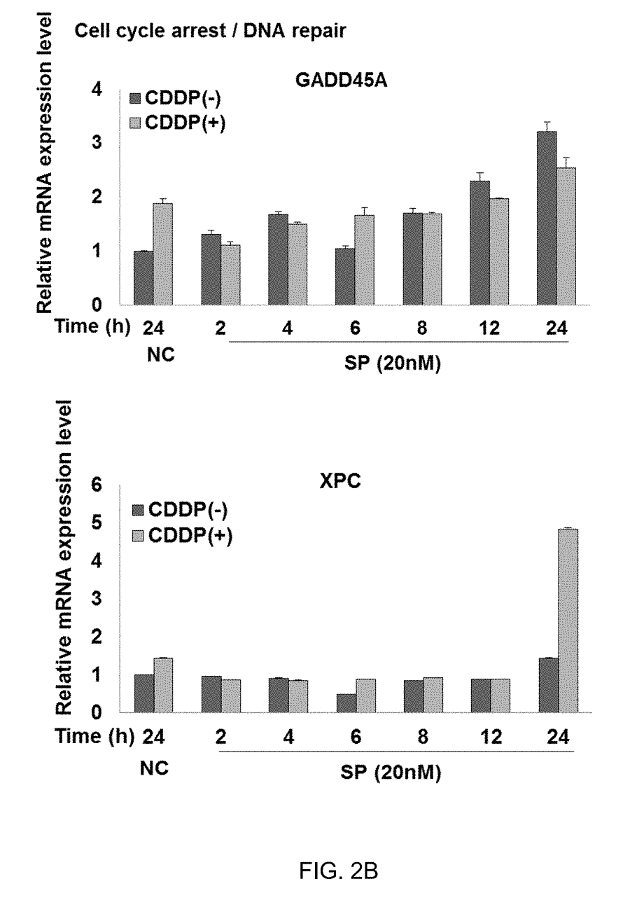

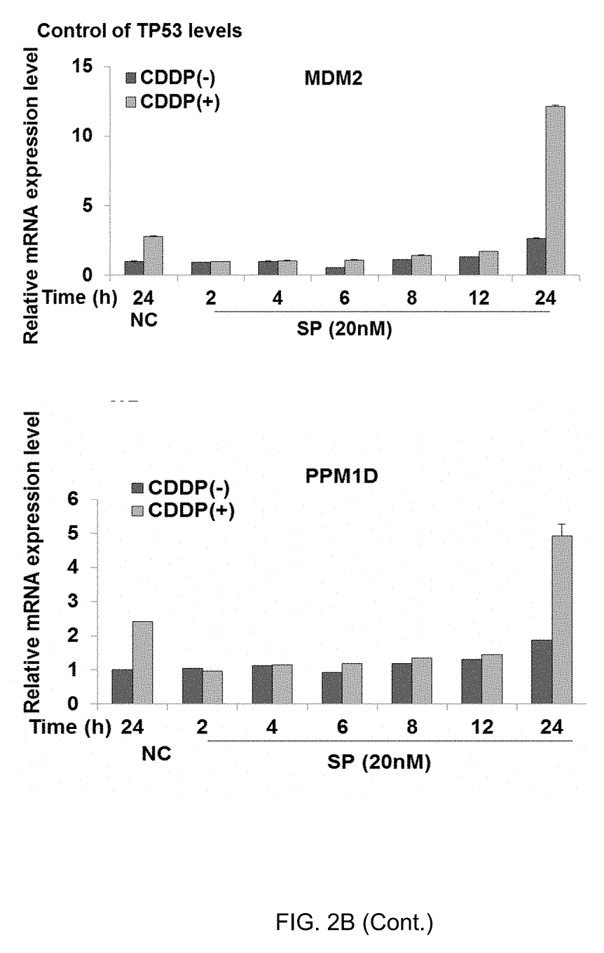

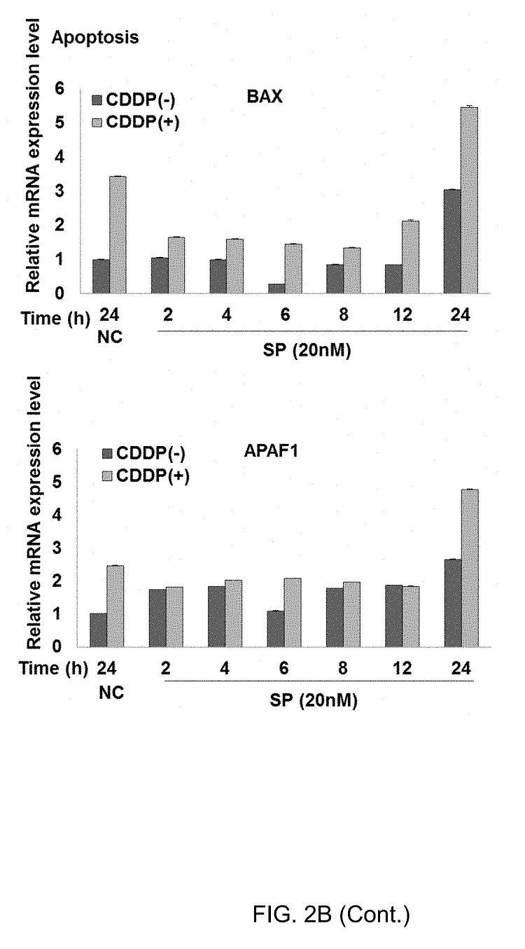

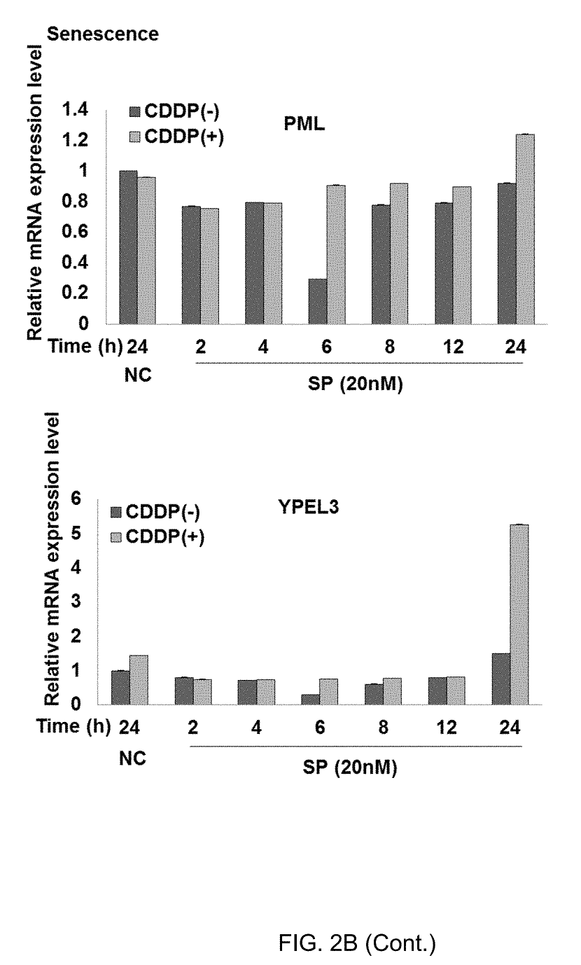

As a result, as shown in FIG. 2, the expression levels of GADD45A, XPC, MDM2, PPM1D, BAX, APAF1, PML and YPEL3 were increased when cisplatin and HPV E6/E7 siRNA were treated together.

### Example 3

Effect of Cisplatin and HPV E6/E7 siRNA on Apoptosis in Cervical Cancer Cells

To investigate the effects of cisplatin (CDDP) and HPV E6/E7 siRNA on the activity of TP53 and apoptosis in cervical cancer cells, the following experiment was conducted.

HeLa and CaSki cells were cultured on a 6-well plate and transformed with a GFP-TP53 vector having a fluorescent substance to construct a stable cell line. The GFP-TP53 vector utilized a lentivirus system with puormycin (Sigma-Aldrich, St. Louis, Mo.) resistance marker.

Cisplatin (CDDP) and HPV E6/E7 siRNA were treated in the above prepared stable cell line. The surviving cancer cells in each well were then photographed for 5 days using an IncuCyte HD system (Essen Instruments, Ann Arbor, Mich.) using the time-lapse technique. In addition, the cell proliferation rate and the number and intensity of GFP, which signify TP53, for each HPV E6/E7 siRNA were analyzed using Incucyte ZOOM software (Essen Bioscience).

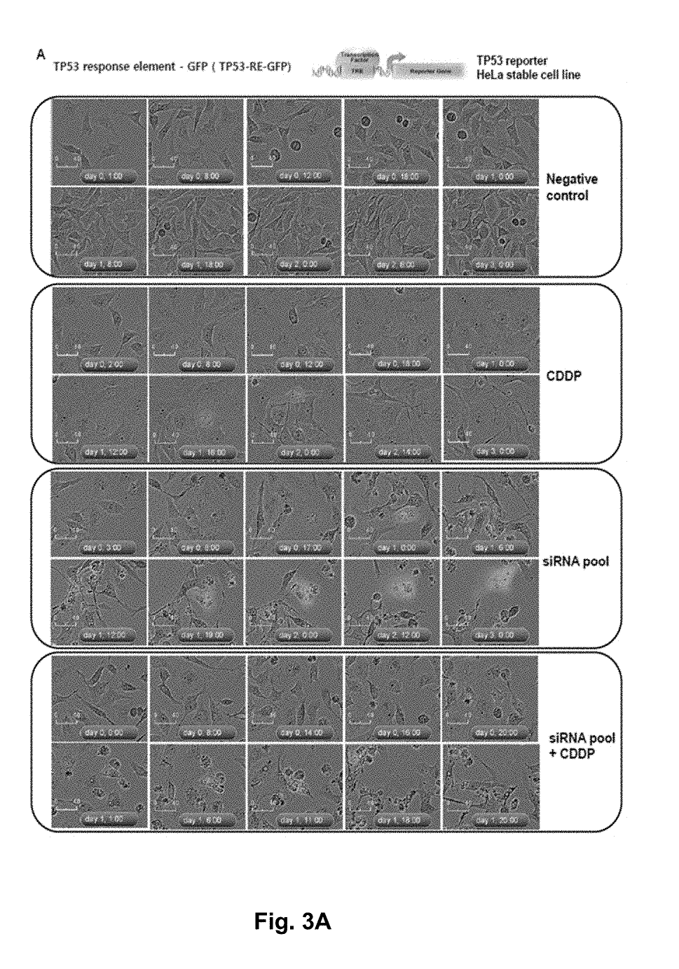

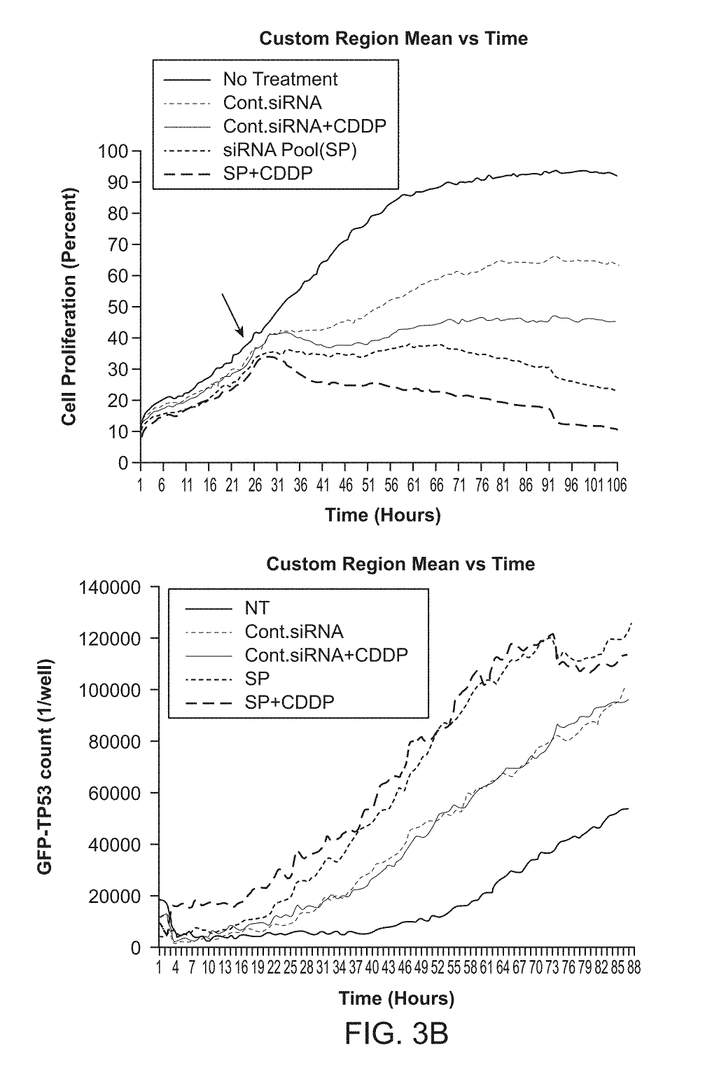

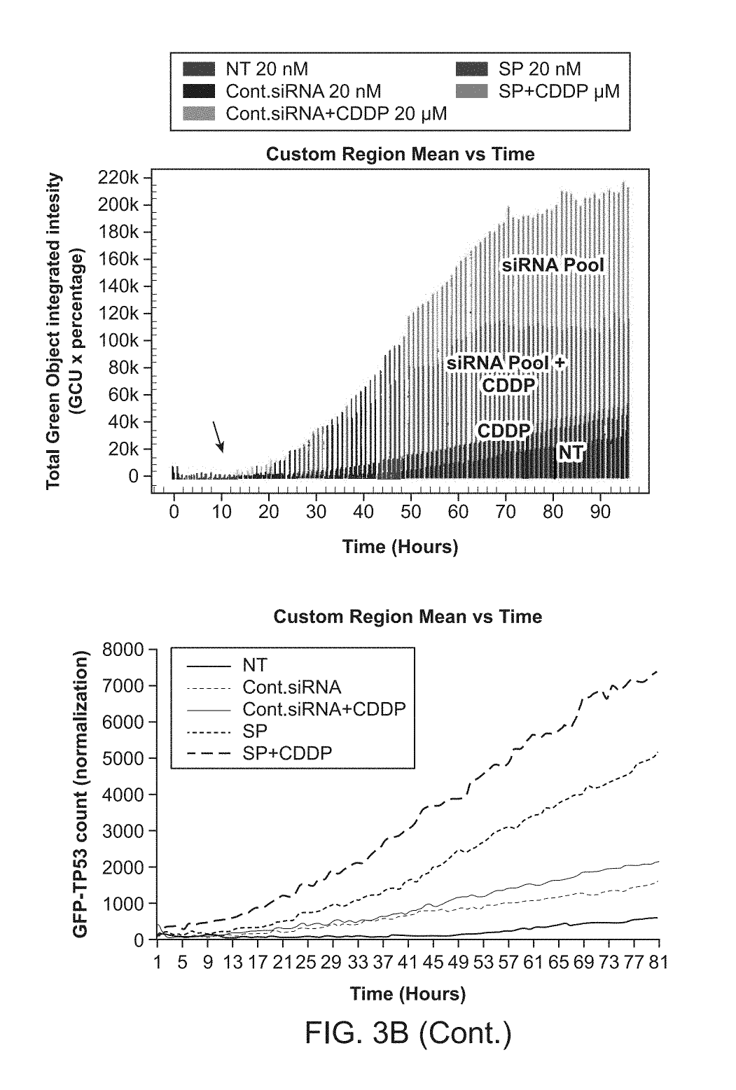

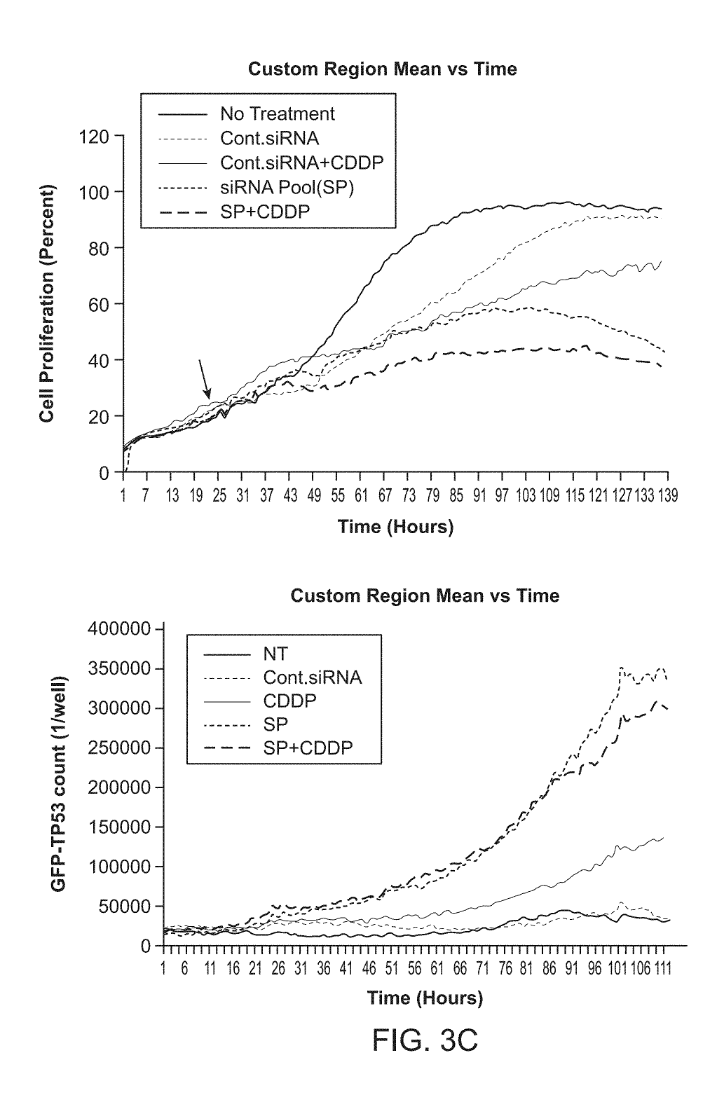

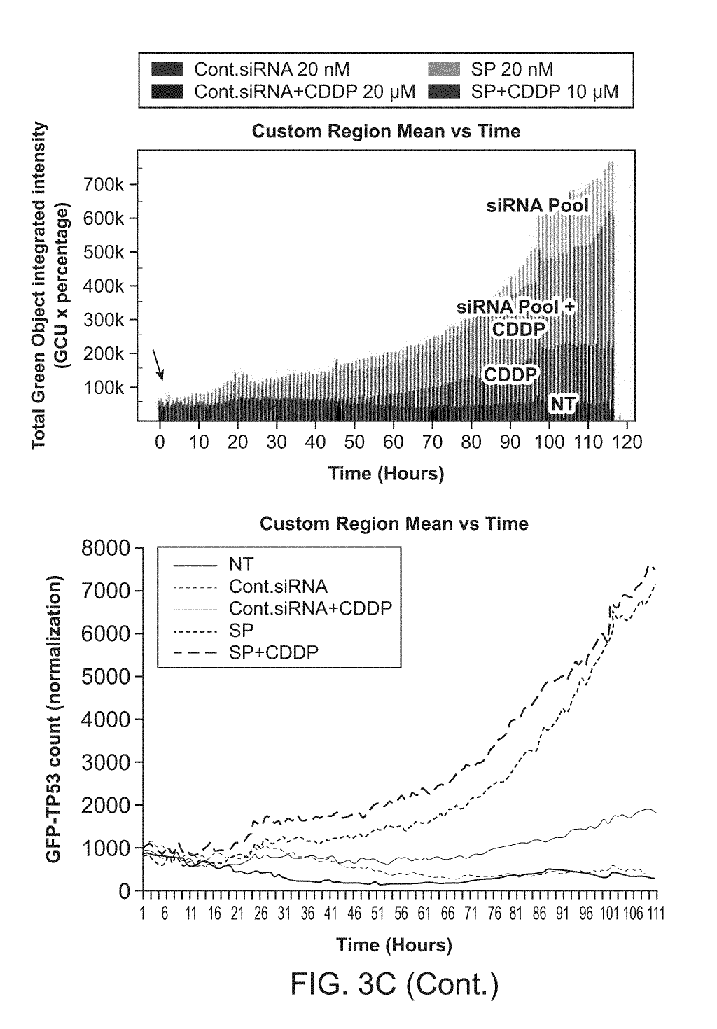

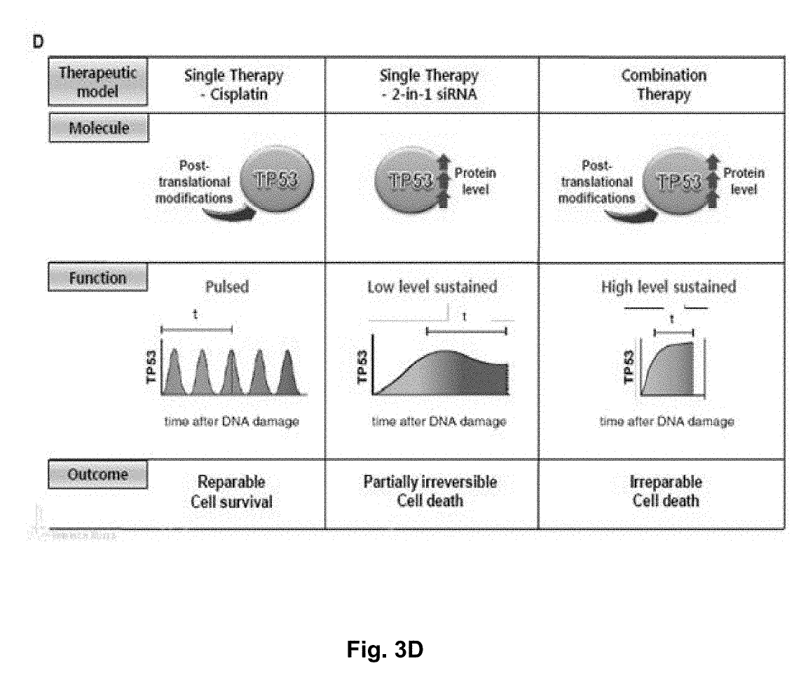

As a result, as shown in FIG. 3, when the siRNA and cisplatin were treated together in Hela cells, the cell proliferation rate was decreased and the number of GFP-bound TP53 was increased with time (FIG. 3A, 3B). Also, in CaSki cells treated with siRNA and cisplatin, the cell proliferation rate decreased, and the number of GFP-bound TP53 was further increased with time (FIG. 3C).

### Example 4

TP53 and E2F in Response to the Binding of Cisplatin to HPV E6/E7 siRNA in Cervical Cancer Cells

To investigate the changes of TP53 and E2F in the binding of cisplatin (CDDP) and HPV E6/E7 siRNA in cervical cancer cells, the following experiment was conducted.

Hela cells were cultured in 96-well plates to transform GFP-conjugated TP53 and RFP-conjugated E2F and then treated with HPV E6/E7 siRNA or cisplatin. Subsequently, changes in GFP-TP53 and RFP-E2F over time in Hela cells with silencing effects of HPV E6/E7 siRNA were recorded using confocal microscopy. Red was taken at Ex/Em=565 nm/650 nm and green was taken at Ex/Em=495 nm/545 nm. The mean intensity of each signal was observed using time-lapse confocal images of stable cells photographed every 20 minutes from 12 hours to 24 hours.

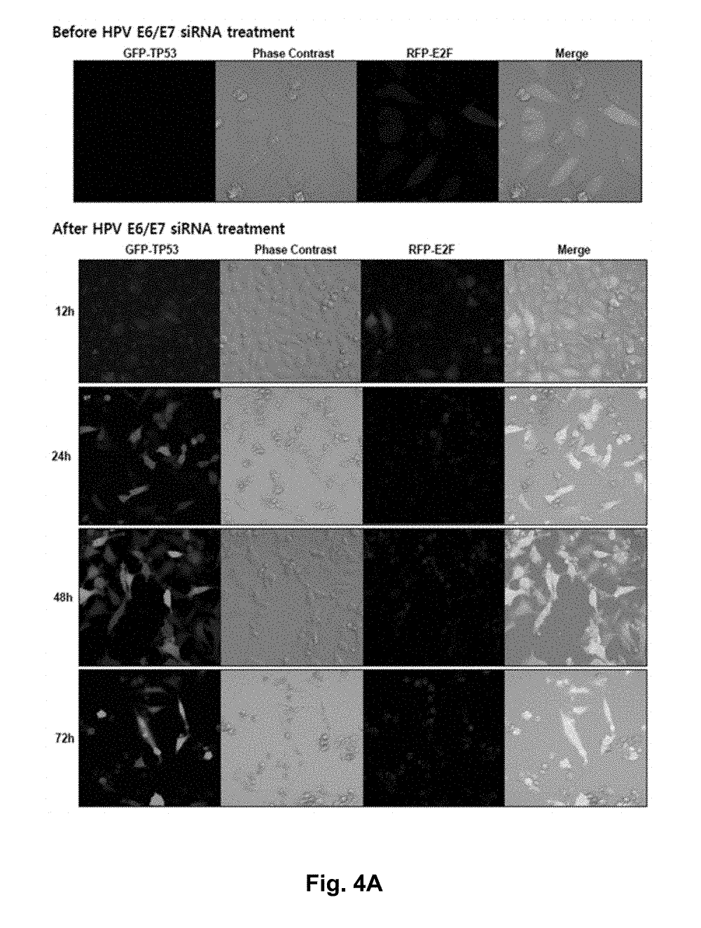

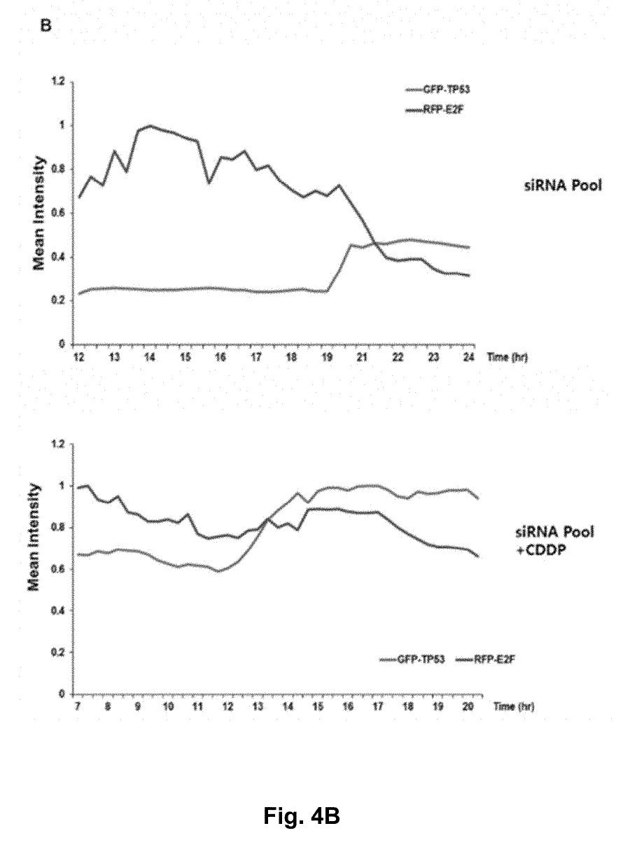

As a result, as shown in FIG. 4, it was confirmed that the expression level of GFP-conjugated TP53 was increased after treatment with HPV E6/E7 siRNA compared with before treatment, and that the green signal was stronger over time. The expression level of GFP-conjugated TP53 increased in 19 to 21 hours, and the amount of expression of RFP-bound E2F was decreased inversely (FIG. 4A).

On the other hand, in the case of the combination treatment of cisplatin (CDDP) and HPV E6/E7 siRNA, the expression level of GFP-bound TP53 increased at 12 to 14 hours, and the expression level of RFP-bound E2F decreased (FIG. 4B).

### Example 5

Therapeutic Effect of Triple Mixtures on HPV Positive Cervical Cancer Cells

### Example 5-1

Therapeutic Effect of Triple Mixtures on HPV Positive Cervical Cancer Cells In Vitro

To investigate the therapeutic effects of treatment with cisplatin (CDDP), HPV E6/E7 siRNA and anticancer drugs in HPV-positive cervical cancer cells, the following experiment was conducted.

Hela cells were treated with HPV E6/E7 siRNA, cisplatin, and paclitaxel (PTX), an anticancer drug. Then cell lysates were obtained and subjected to Western blotting, and RNA was extracted and subjected to RT-qPCR. Sequences of primers and probes are as follows, all of which were produced in TIB MOLBIOL (Berlin, Germany).

CDKN1A forward
5′-CGA AGT CAG TTC CTT GTG GAG-3′,
CDKN1A reverse
5′-CAT GGG TTC TGA CGG ACA T-3′,
TaqMan probe
5′-FAM-CAG AGG AG-Dark quencher-3′

mRNA levels were determined at 530 and 705 nm wavelengths using the LightCycler Real-time PCR Detection System (Roche Diagnostics, Basel, Switzerland).

In addition, the cell proliferation rate, number and intensity of GFP were measured using an IncuCyte HD system (Essen Instruments, Ann Arbor, Mich.) using GFP-conjugated TP53. In addition, Hela cells were treated with HPV E6/E7 siRNA, cisplatin and cisplatin+paclitaxel, or a mixture thereof, followed by cell cycle analysis via FACS assay and the percentages (% cells) of cells in each step (G0-G1, S and G2/M) were measured.

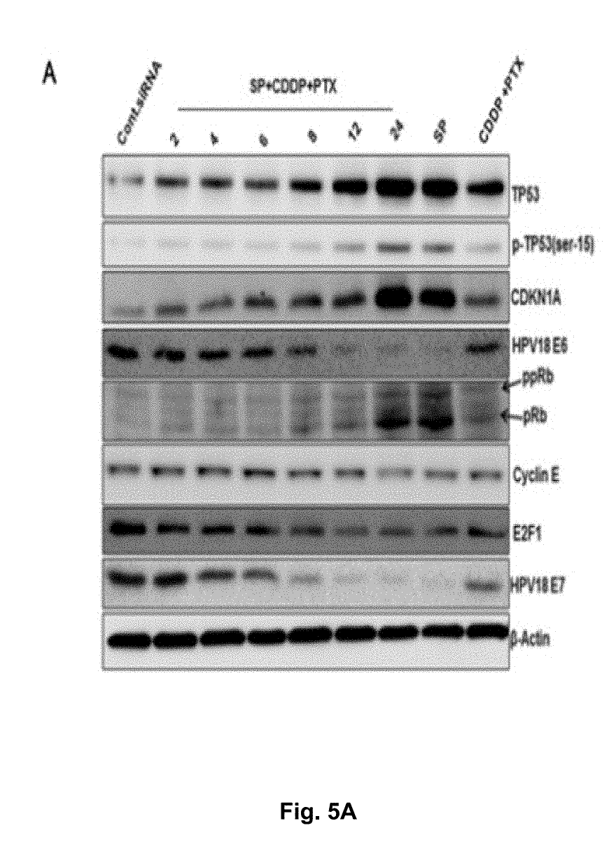

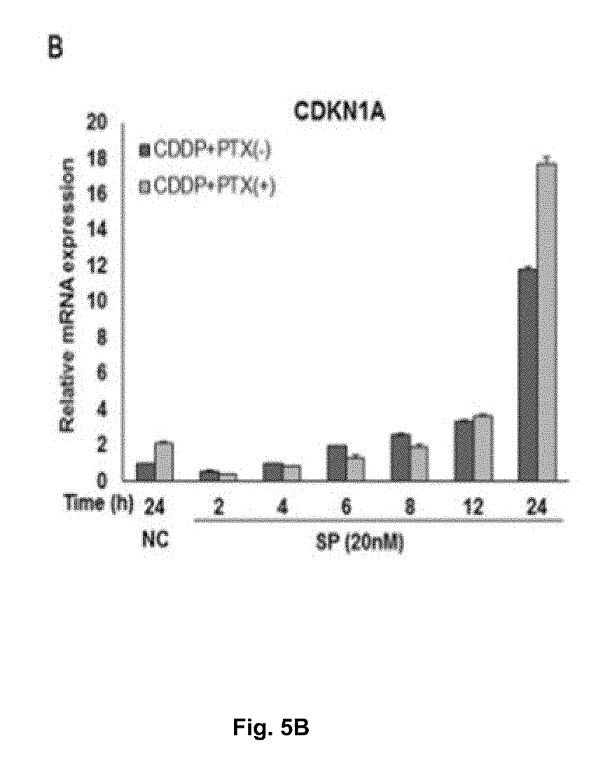

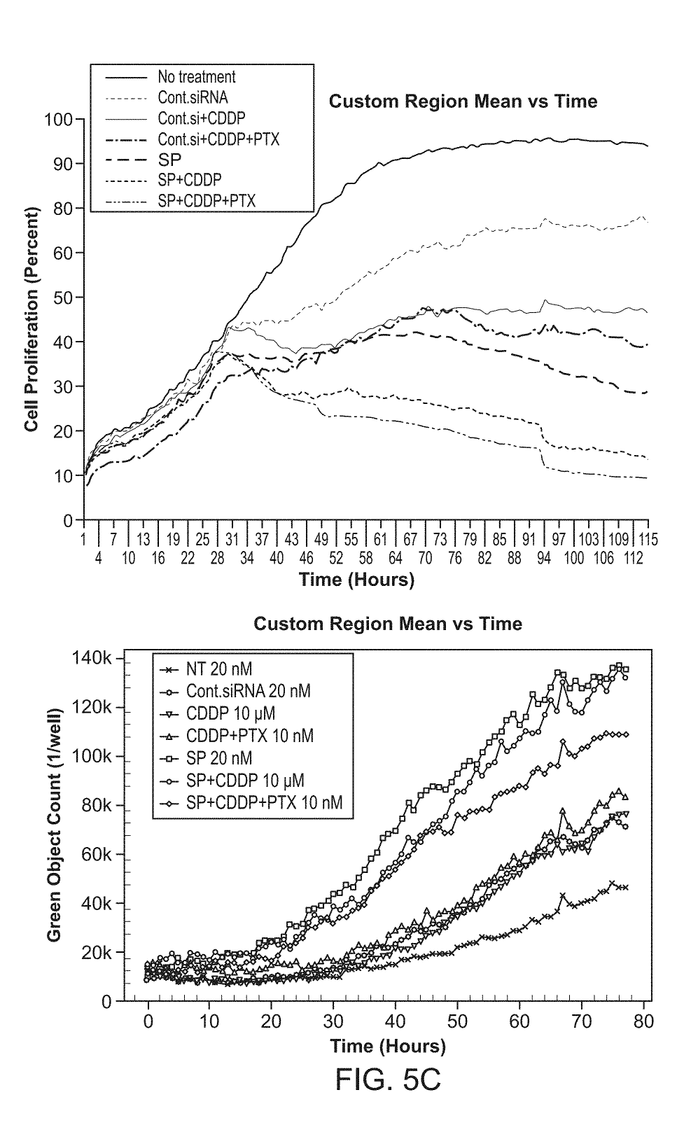

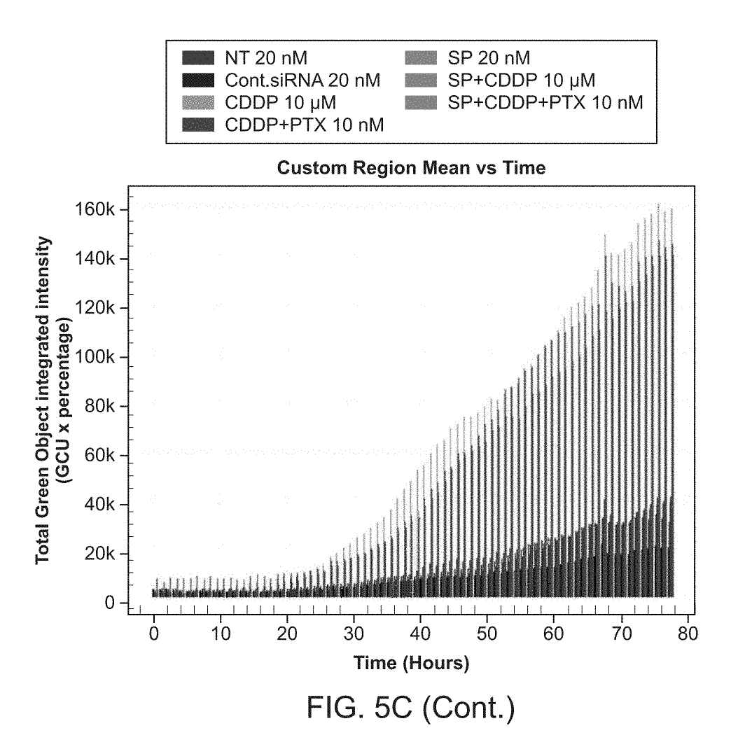

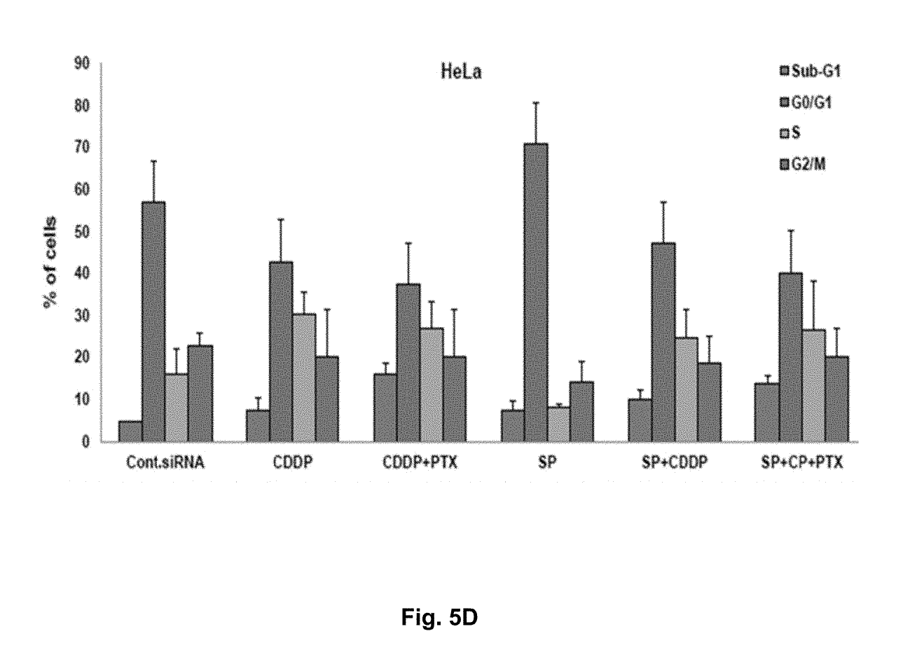

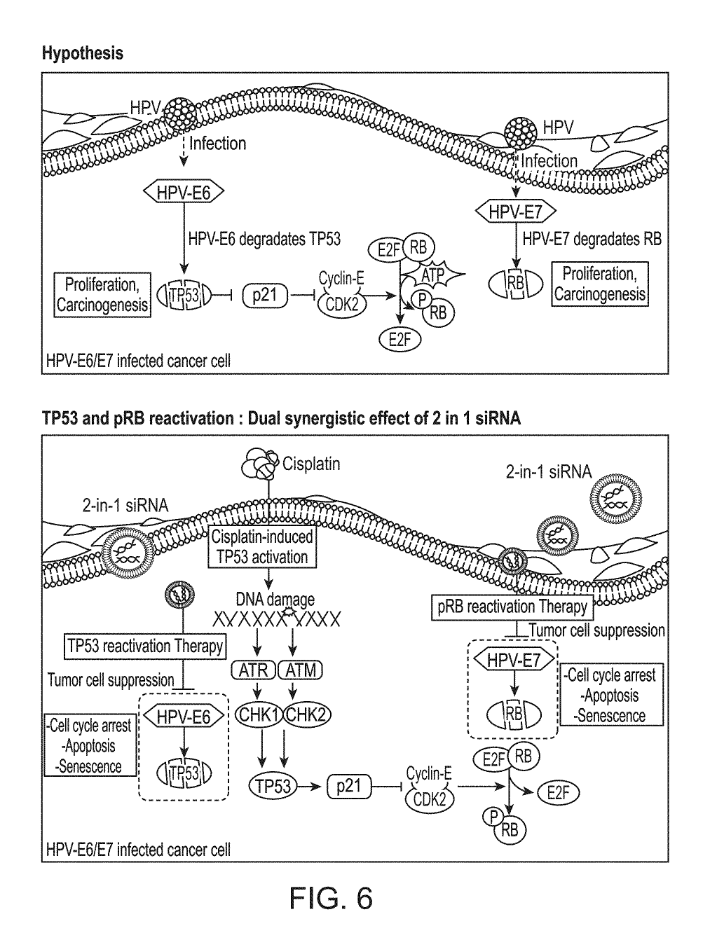

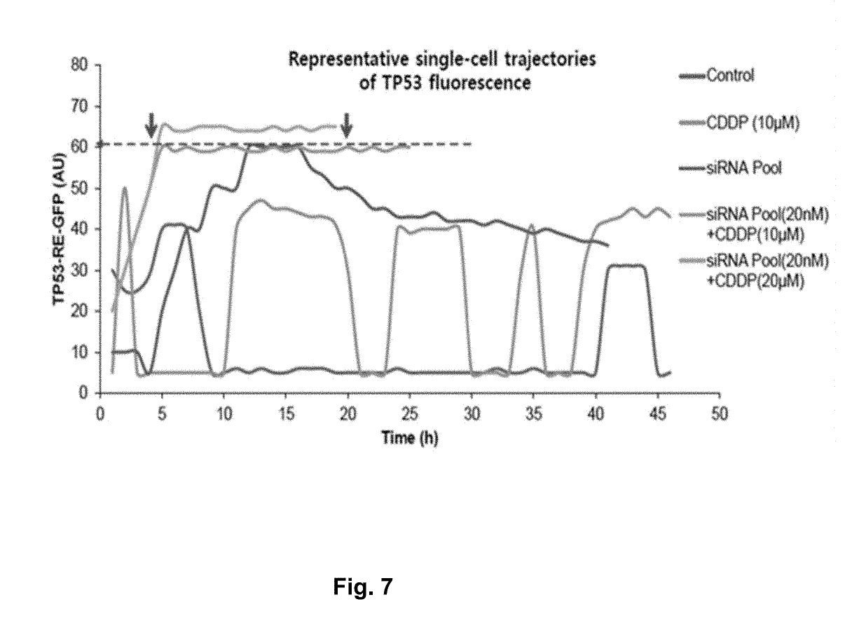

The results are shown in FIGS. 5A to 5D.

When HeLa cells were treated with HPV E6/E7 siRNA, cisplatin, and paclitaxel, protein expression of TP53 increased over time, but protein expression of 18-E6 and 18-E7 decreased (FIG. 5A). In addition, when Hela cells were treated with HPV E6/E7 siRNA pool (SP, 20 nM), cisplatin (5 μM), and paclitaxel (10 nM) together, the expression level of CDKN1A mRNA was increased (FIG. 5B). In addition, When Hela cells, transformed with GFP-TP53, were treated with the three mixtures, cell proliferation was inhibited, the number of GFP was increased, and the intensity of GFP was increased (FIG. 5C).

On the other hand, when the Hela cells were treated with HPV E6/E7 siRNA pool (SP, 20 nM), cisplatin (5 μM), and paclitaxel (10 nM), the proportion of Sub-G1 phase cells was high and the ratio of G0/G1 phase cells was low. This confirms that the mixtures of HPV E6/E7 siRNA, cisplatin, and paclitaxel inhibit the cell cycle of G1 and G2/M.

### INDUSTRIAL AVAILABILITY

The present invention is to provide a method of maintaining elevated levels of p53 in cells, by administering a platinum-based anticancer drug and an siRNA against ubiquitin ligase to p53 to a subject in need thereof, in combination or sequentially. The method according to the present invention makes it possible to maintain the increased expression level of intracellular p53 for a long period of time even after treatment with a low concentration of platinum-based anticancer drug, thereby effectively inducing the death of cancer cells and minimizing the side effects of drug administration upon administration of the platinum-based anticancer drug, and can be usefully used for the development of a preventive or therapeutic agent for cancer, thus being highly industrially applicable.

1. A method for maintaining an elevated levels of p53 in a cell, the method comprising administering a platinum-based anticancer drug and a siRNA against ubiquitin ligase to p53 to a subject in need thereof, in combination or sequentially.

2. The method according to claim 1, wherein the platinum-based anticancer drug is selected from the group consisting of cisplatin (cis-diamminedichloroplatinum [II]), carboplatin, oxaliplatin, nedaplatin, picoplatin, triplatin tetranitrate, satraplatin, and mixtures thereof.

3. The method according to claim 1, wherein the ubiquitin ligase is selected from the group consisting of E6/E6-AP complex of Human Papillomavirus (HPV), E6, E6-AP, human HDM2, Pirh2 and COP1.

4. The method according to claim 1, wherein the cell is a cancer cell of the subject.

5. The method of claim 1, wherein the method induces the death of the cell.

6. The method of claim 5, wherein the death of the cell is induced via apoptosis pathway.

7. The method of claim 1, wherein the siRNA is selected from the group consisting of SEQ ID NOs: 1 to 10.

8. A composition for promoting apoptosis by maintaining an elevated level of p53 in a cell, comprising, as an active ingredient, a platinum-based anticancer drug and a siRNA against ubiquitin ligase to p53.

9. The composition of claim 8, wherein the cell is a cancer cell.

10. A pharmaceutical composition for preventing or treating cancer by maintaining an elevated levels of p53 in a cancer cell, comprising, as an active ingredient, a platinum-based anticancer drug and an siRNA against ubiquitin ligase to p53.

11. A method for screening a preventive or therapeutic agent of cervical cancer, the method comprising steps of:
(a) transducing a cervical cancer cell line with a siRNA against E6 and E7 of Human Papillomavirus virus (HPV);
(b) treating the cell line transduced in the step (a) with a cervical cancer therapeutic candidate substance;
(c) measuring the expression level of intracellular p53 (tumor protein 53) at regular intervals for up to 48 hours from immediately after therapeutic candidate substance is treated; and
(d) selecting a substance having a prolonged increase in the expression level of intracellular p53 (tumor protein 53) as compared to a cell not treated with a therapeutic candidate substance.

12. The method according to claim 11, further comprising the step of:
(e) after the step (d), confirming the effect of the selected substance in a cell or an animal.

13. The method of claim 11, wherein the expression of p53 in the step (c) is measured by a method selected from the group consisting of RT-PCR, competitive RT-PCR, real-time RT-PCR, RNase protection assay (RPA), Northern blotting, DNA microarray chip analysis, Western blotting, enzyme-linked immunosorbent assay, radioimmunoassay (RIA), radioimmunodiffusion, Ouchterlony immunodiffusion, rocket immunoelectrophoresis, Immunohistochemistry, immunoprecipitation, complement fixation, flow cytometry (FACS) and protein chip analysis.

14. The method according to claim 11, wherein the siRNA is selected from the group consisting of SEQ ID NOs: 1 to 10.

15. The method according to claim 11, wherein the regular intervals of the step (c) is 30 minutes to 1 hour.
